(19) United States
(12) Patent Application Publication
(10) Pub. No.: US 2003/0050305 A1
(43) Pub. Date: Mar. 13, 2003

(54) Thromboxane inhibitors, compositions and methods of use

(76) Inventor: Inigo Saenz de Tejada, Madrid (ES)

Correspondence Address: EDWARD D GRIEFF, HALE & DORR LLP, 1455 PENNSYLVANIA AVE, NW, WASHINGTON, DC, 20004, US

(21) Appl. No.: 10/285,620

(22) Filed: Nov. 1, 2002

Related U.S. Application Data
(63) Continuation of application No. PCT/US01/16318, filed on May 22, 2001.
(60) Provisional application No. 60/205,536, filed on May 22, 2000.

(51) Int. Cl. A61K 31/55; A61K 31/44; A61K 31/4439; A61K 31/195; A61K 31/403

(52) U.S. Cl. 514/217.04; 514/277; 514/411; 514/317; 514/341; 514/567

(57) ABSTRACT

The present invention describes methods for treating or preventing sexual dysfunctions in males and females, and for enhancing sexual responses in males and females by administering a therapeutically effective amount of at least one thromboxane inhibitor, and, optionally, at least one compound that donates, transfers or releases nitric oxide, elevates endogenous levels of endothelium-derived relaxing factor, stimulates endogenous synthesis of nitric oxide or is a substrate for nitric oxide synthase, and/or at least one vasoactive agent. The male or female may preferably be diabetic. The present invention also provides novel compositions comprising at least one thromboxane inhibitor, and, at least one compound that donates, transfers or releases nitric oxide, elevates endogenous levels of endothelium-derived relaxing factor, stimulates endogenous synthesis of nitric oxide or is a substrate for nitric oxide synthase, and, optionally, at least one therapeutic agent, such as, vasoactive agents, nonsteroidal antiinflanmmatory compounds (NSAIDs), selective cyclooxygenase-2 (COX-2) inhibitors, anticoagulants, angiotensin converting enzymes (ACE) inhibitors, angiotensin II receptor antagonists, renin inhibitors, and mixtures thereof. The present invention also provides methods for treating or preventing ischemic heart disorders, myocardial infarction, angina pectoris, stroke, migraine, cerebral hemorrhage, cardiac fatalities, transient ischaemic attacks, complications following organ transplants, coronary artery bypasses, angioplasty, endarterectomy, atherosclerosis, pulmonary embolism, bronchial asthma, bronchitis, pneumonia, circulatory shock of various organs, nephritis, graft rejection, cancerous metastases, pregnancy-induced hypertension, preeclampsia, eclampsia, thrombotic and thromboembolic disorders, intrauterine growth, gastrointestinal disorders, renal diseases and disorders, disorders resulting from elevated uric acid levels and dysmenorrhea, and for inhibiting platelet aggregation or platelet adhesion or relaxing smooth muscles.

### RELATED APPLICATIONS

This application is a continuation of PCT/US01/16318 filed May 22, 2001, which claims priority to U.S. Provisional Application No. 60/205,536 filed May 22, 2000.

### FIELD OF THE INVENTION

The present invention describes methods for treating or preventing sexual dysfunctions in males and females, and for enhancing sexual responses in males and females by administering a therapeutically effective amount of at least one thromboxaiie inhibitor, and, optionally, at least one compound that donates, transfers or releases nitric oxide, elevates endogenous levels of endothelium-derived relaxing factor, stimulates endogenous synthesis of nitric oxide or is a substrate for nitric oxide synthase, and/or at least one vasoactive agent. The male or female may preferably be diabetic. The present invention also provides novel compositions comprising at least one thromboxane inhibitor, and, at least one compound that donates, transfers or releases nitric oxide, elevates endogenous levels of endothelium-derived relaxing factor, stimulates endogenous synthesis of nitric oxide or is a substrate for nitric oxide synthase, and, optionally, at least one therapeutic agent, such as, vasoactive agents, nonsteroidal antiinflammatory compounds (NSAIDs), selective cyclooxygenase-2 (COX-2) inhibitors, anticoagulaits, angiotensin converting enzymes (ACE) inhibitors, angiotensin II receptor antagonists, renin inhibitors, and mixtures thereof. The present invention also provides methods for treating or preventing ischemic heart disorders, myocardial infarction, angina pectoris, stroke, migraine, cerebral hemorrhage, cardiac fatalities, transient ischaemic attacks, complications following organ transplants, coronary artery bypasses, angioplasty, endarterectomy, atherosclerosis, pulmonary embolism, bronchial asthma, bronchitis, pneumonia, circulatory shock of various organs, nephritis, graft rejection, cancerous metastases, pregnancy-induced hypertension, preeclampsia, eclampsia, thrombotic and thromboembolic disorders, intrauterine growth, gastrointestinal disorders, renal diseases and disorders, disorders resulting from elevated uric acid levels and dysmenorrhea, and for inhibiting platelet aggregation or platelet adhesion or relaxing smooth muscles.

### BACKGROUND OF THE INVENTION

Adequate sexual function is a complex interaction of hormonal events and psychosocial relationships. There are four stages to sexual response as described in the International Journal of Gynecology & Obstetrics, 51(3):265-277 (1995). The first stage of sexual response is desire. The second stage of sexual response is arousal. Both physical and emotional stimulation may lead to breast and genital vasodilation and clitoral engorgement (vasocongestion). In the female, dilation and engorgement of the blood vessels in the labia and tissue surrounding the vagina produce the “orgasmic platform,” an area at the distal third of the vagina where blood becomes sequestered. Localized perivaginal swelling and vaginal lubrication make up the changes in this stage of sexual response. Subsequently, ballooning of the proximal portion of the vagina and elevation of the uterus occurs. In the male, vasodilation of the cavernosal arteries and closure of the venous channels that drain the penis produce an erection. The third stage of sexual response is orgasm, while the fourth stage is resolution. Interruption or absence of any of the stages of the sexual response cycle can result in sexual dysfunction. One study found that 35% of males and 42% of females reported some form of sexual dysfunction. Read et al, J. Public Health Med., 19(4):387-391 (1997).

While there are obvious differences in the sexual response between males and females, one common aspect of the sexual response is the erectile response. The erectile response in both males and females is the result of engorgement of the erectile tissues of the genitalia with blood which is caused by the relaxation of smooth muscles in the arteries serving the genitalia.

In males, some pharmacological methods of treatment are available, however, such methods have not proven to be highly satisfactory or without potentially severe side-effects. Papaverine is now widely used to treat impotence. Papaverine is generally effective in cases where the dysfunction is psychogenic or neurogenic and where severe atherosclerosis is not involved. Injection of papaverine, a smooth muscle relaxant, or phenoxybenzamine, a non-specific antagonist and hypotensive, into a corpus cavernosum has been found to cause an erection sufficient for vaginal penetration, however, these treatments are not without the serious and often painful side effect of priapism. Also, in cases where severe atherosclerosis is not a cause of the dysfunction, intracavernosal injection of phentolamine, an α-adrenergic antagonist, is used. As an alternative or, in some cases, as an adjunct to α-adrenergic blockade, prostaglandin E₁ (PGE₁) has been administered via intracavemosal injection. A major side effect frequently associated with intracorprally-delivered PGE₁ is penile pain and burning.

Thromboxane A₂ is an extremely potent, short-lived endogenous mediator that induces both platelet activation and aggregation and smooth muscle contraction. It is produced by the action of the enzyme thromboxane synthase on the prostaglandin endoperoxide PGH₂, which itself is a potent thromboxane receptor agonist. Thromboxane A₂ has been implicated as a potential contributor in the pathogenesis of numerous diseases and disorders. Thromboxane A₂ receptor antagonists, thromboxane synthase inhibitors and dual acting thromboxane receptor antagonist/synthase inhibitors have been developed to treat numerous diseases. For example, U.S. Pat. Nos. 5,021,443, 5,128,359, 5,296,494 and 5,496,849 describe treating ischemic heart disorders, myocardial infarction, angina pectoris, stroke, cerebral hemorrhage, atherosclerosis, pulmonary embolism, bronchial asthma, bronchitis, pneumonia, circulatory shock of various organs, such as, for example, hemorrhage, septicemia, heart failure, trauma, acute pancreatitis, burn and bacterial origin, nephritis, graft rejection and cancerous metastases with thromboxane A₂ receptor antagonists; U.S. Pat. Nos. 4,839,384 and 5,021,443 and WO 99/45905 describe treating migraines with thromboxane A₂ receptor antagonists; U.S. Pat. No. 5,312,818 describes treating gastrointestinal disorders; U.S. Pat. No. 4,968,673 describes the use of thromboxane receptor antagonists to treat renal diseases and disorders; U.S. Pat. No. 4,968,703 discloses methods to treat occlusive vascular diseases; U.S. Pat. No. 5,021,448 discloses methods to treat disorders that result from elevated uric acid levels with thromboxane synthase inhibitors, thromboxane receptor antagonists and/or combinations thereof; U.S. Pat. No. 5,605,917 discloses methods of treating dysmenorrhea with thromboxane A₂ receptor antagonists alone or in combination with NSAIDs; U.S. Pat. No. 5,015,648 describes the use of thromboxane receptor antagonists and thromboxane synthase inhibitors for the treatment of pregnancy-induced hypertension, preeclampsia, eclampsia and reduction of intrauterine growth; U.S. Pat. No. 5,532,321 discloses the use of thromboxane antagonists in combination with prostacyclins, their analogs and/or prostaglandins for the treatment of thrombotic and thromboembolic syndromes; and Circulation, 81, suppl I, 1-69 (1990) and U.S. Pat. No. 5,496,849 describe inhibiting platelet aggregation or platelet adhesion with thromboxane receptor antagonists. The disclosures of each of these patents, applications and publications are incorporated by reference herein in their entirety.

There is a need in the art for new and improved treatments of sexual dysfunctions, and other diseases. The present invention is directed to these, as well as other, important ends.

### SUMMARY OF THE INVENTION

In arriving at the present invention, it was unexpectedly discovered that the relaxation of the trabecular smooth muscle is mediated by the thromboxane receptor. Thromboxane A₂ is overproduced in diabetic patients who also tend to have a greater incidence of sexual dysfunctions. Blocking of the thromboxane receptor results in the relaxation of the trabecular smooth muscle, thereby suggesting a therapy for the treatment of sexual dysfunctions. Additionally, NO and NO donors have been implicated as mediators of nonvascular smooth muscle relaxation. As described herein, this effect includes the dialation of the corpus smooth muscle, an event involved in the sexual response process in both males and females. The smooth muscle relaxant properties of the thromboxane inhibitors and of compounds that donates, transfers or releases nitric oxide, elevates endogenous levels of endothelium-derived relaxing factor (EDRF), stimulates endogenous synthesis of nitric oxide or is a substrate for nitric oxide synthase work together to permit the same efficacy with lower doses of the thromboxane inhibitors or work synergistically to produce an effect that is greater than the additive effects of the thromboxane inhibitor and the compound that donates, releases or transfers nitrogen monoxide, elevate levels of endogenous nitric oxide or EDRF or is a substrate for nitric oxide synthase. The thromboxane inhibitor and nitric oxide donors may be administered separately or as components of the same composition in one or more pharmaceutically acceptable carriers.

One aspect of the present invention describes methods for preventing or treating sexual dysfunctions or enhancing sexual responses in patients, including males and females, by administering to a patient in need thereof a therapeutically effective amount of at least one thromboxane inhibitor, and, optionally, at least one compound that donates, transfers or releases nitric oxide, elevates endogenous levels of endothelium-derived relaxing factor, stimulates endogenous synthesis of nitric oxide or is a substrate for nitric oxide synthase. Tie methods can further comprise administering a therapeutically effective amount of at least one vasoactive agent. Alternatively, the methods for treating and/or preventing sexual dysfunctions and/or enhancing sexual responses in patients, including males and females, can comprise administering a therapeutically effective amount of at least one thromboxane inhibitor, at least one vasoactive agent, and, optionally, at least one compound that donates, transfers or releases nitric oxide as a charged species, i.e., nitrosonium (NO⁺) or nitroxyl (NO), or as the neutral species, nitric oxide (NO.), and/or stimulates endogenous production of nitric oxide or EDRF in vivo and/or is a substrate for nitric oxide synthase. The patient may preferably be diabetic. The thromboxane inhibitors, the nitric oxide donors, and the vasoactive agents may be administered separately or as components of the same composition in one or more pharmaceutically acceptable carriers.

Another aspect of the invention provides compositions comprising at least one thromboxane inhibitor and at least one compound that donates, transfers or releases nitric oxide as a charged species, i.e., nitrosonium (NO⁺) or nitroxyl (NO−), or as the neutral species, nitric oxide (NO.), and/or stimulates endogenous production of nitric oxide or EDRF in vivo and/or is a substrate for nitric oxide synthase, and, optionally, at least one therapeutic agent.

Yet another aspect of the present invention provides methods using the compositions described herein for treating and/or preventing ischemic heart disorders, myocardial infarction, angina pectoris, stroke, migraine, cerebral hemorrhage, atherosclerosis, pulmonary embolism, bronchial asthma, bronchitis, pneumonia, circulatory shock of various organs, nephritis, graft rejection, cancerous metastases, pregnancy-induced hypertension, preeclampsia, eclampsia, thrombotic and thromboembolic disorders, intrauterine growth, gastrointestinal disorders, renal diseases and disorders, disorders resulting from elevated uric acid levels, or dysmenorrhea, and for inhibiting platelet aggregation or platelet adhesion or relaxing smooth muscles. In these methods, the thromboxane inhibitors, nitric oxide donors and/or therapeutic agents may be administered separately or as components of the same composition in one or more pharmaceutically acceptable carriers.

These and other aspects of the present invention are described in detail herein.

### BRIEF DESCRIPTION OF THE FIGURES

FIGS. 1A and 1B are concentration response curves of human corpus cavernosum tissue for PGE₁ or PGE₀ in the absence or presence of the thromboxane A₂ receptor antagonist, SQ 29548. Tissues were incubated in a physiological salt solution, bubbled with 95% O₂, contracted with 1 μM phenylephrine, and then treated with increasing concentrations of PGE₁ alone (open circles); or increasing concentrations of PGE₀ (closed circles) in the absence (FIG. 1A) or presence of 0.02 μM SQ 29548 (FIG. 1B). n is the number of samples measured for each condition tested. In the x-axis, log M [drugs] corresponds to ten fold increases of PGE₁ or PGE₀ from 0.001 μM (at −9) to 100 μM (at −4). Data are expressed as mean±standard error of the percentage of total relaxation induced by 100 μM papaverine hydrochloride. P<0.01 by two-factor ANOVA analysis using StatView software for Apple computers.

FIG. 2 is a concentration response curve of human corpus cavernosum tissue for PGE₀ in the absence or presence of the thromboxane A₂ receptor antagonist, SQ 29548. Tissues were incubated in a physiological salt solution, bubbled with 95% O₂, contracted with 1 μM phenylephrine, and then treated with increasing concentrations of PGE₀ alone (open circles), where a total of 17 samples were tested (n=17); or increasing concentrations of PGE₀ in the presence of 0.02 μM SQ 29548 (closed circles), where a total of 6 samples were tested (n=6). In the x-axis, log M [PGE₀] corresponds to ten fold increases of PGE₀ from 0.001 μM (at −9) to 100 μM (at −4). Data are expressed as mean±standard error of the percentage of total relaxation induced by 100 μM papaverine hydrochloride. P<0.01 by two-factor ANOVA analysis using StatView software for Apple computers.

FIG. 3 is a concentration response curve of human corpus cavernosum tissue for PGE₁ in the absence or presence of the thromboxane A₂ agonist, U46619. Tissues were incubated in a physiological salt solution, bubbled with 95% O₂, contracted with 1l M phenylephrine, and then treated with increasing concentrations of PGE₁ alone (control, open circles); or increasing concentrations of PGE₁ in the presence of 0.003 μM U46619 (closed circles), where a total of 6 samples were tested for each condition (n=6). In the x-axis, log M [PGE₁] corresponds to ten fold increases of PGE₁ from 0.001 μM (at −9) to 100 μM (at −4). Data are expressed as mean±standard error of the percentage of total relaxation induced by 100 μM papaverine hydrochloride. P<0.01 by two-factor ANOVA analysis using StatView software for Apple computers.

FIGS. 4A and 4B are concentration response curves of human corpus cavernosum tissue obtained from diabetic or non-diabetic patients for PGE₁ or PGE₀. Tissues from non-diabetic patients (open circles) or diabetic patients (closed circles) were incubated in a physiological salt solution, bubbled with 95% O₂, contracted with 1 μM phenylephrine, and then treated with increasing concentrations of PGE₁ alone (FIG. 4A); or increasing concentrations of PGE₀ alone (FIG. 4B). n is the number of samples measured for each condition tested. In the x-axis, log M [PGE₁] or log M [PGE₀] corresponds to ten fold increases of PGE₁ or PGE₀ respectively, from 0.001 μM (at −9) to 100 μM (at −4). Data are expressed as mean±standard error of the percentage of total relaxation induced by 100 μM papaverine hydrochloride. P<0.01 by two-factor ANOVA analysis using StatView software for Apple computers.

FIG. 5 is a concentration response curve of human corpus cavernosum tissue obtained from diabetic or non-diabetic patients for PGE₀ in the presence of the thromboxane A₂ receptor antagonist, SQ 29548. Tissues from non-diabetic patients (open circles) where a total of 3 samples were tested (n=3); or diabetic patients (closed circles) where a total of 4 samples were tested (n=4); were incubated in a physiological salt solution, bubbled with 95% O₂, contracted with 1 μM phenylephrine, and then treated with increasing concentrations of PGE₀. In the x-axis, log M [PGE₀] corresponds to ten fold increases of PGE₀ , from 0.001 μM (at −9) to 100 μM (at −4). Data are expressed as mean±standard error of the percentage of total relaxation induced by 100 μM papaverine hydrochloride.

FIG. 6 is the concentration contraction response curves of human corpus cavernosum tissue from diabetic and non-diabetic patients in the presence of the thromboxane A₂ receptor agonist, U 46619. Tissues from non-diabetic patients (open circles) or diabetic patients (closed circles) were incubated in a physiological salt solution, bubbled with 95% O₂, contracted with 1 μM phenylephrine, and then treated with increasing concentrations of U 46619. n is the number of samples measured for each condition tested. In the x-axis, log M [U46619] corresponds to ten fold increases of U 46619 from 0.01 M (at −11) to 10 μM (at −5). Data are expressed as mean±standard error of the percentage of maximum contraction induced by U 46691. P<0.01 by two-factor ANOVA analysis using StatView software for Apple computers.

FIG. 7 is the concentration response curves of human corpus cavernosum tissue obtained from hypertensive (HTA) or non-hypertensive patients for PGE₀. Tissues from non-hypertensive patients (open circles) or hypertensive patients (closed circles) were incubated in a physiological salt solution, bubbled with 95% O₂, contracted with 1 μM phenylephrine, and then treated with increasing concentrations of PGE₀. n is the number of samples measured for each condition tested. In the x-axis, log M [PGE₀] corresponds to ten fold increases of PGE₀ from 0.001 μM (at −9) to 10 μM (at −5). Data are expressed as mean±standard error of the percentage of total relaxation induced by 100 μM papaverine hydrochloride. P<0.01 by two-factor ANOVA analysis using StatView software for Apple computers.

FIG. 8 is a concentration response curve of human corpus cavernosum resistance arteries for acetylcholine (ACh) in the absence or presence of the thromboxane A₂ receptor antagonist, SQ 29548. Segments were incubated in a physiological salt solution, bubbled with 95% O₂, contracted with 1 μM norepinephrine, and then treated with increasing concentrations of acetylcholine alone (open circles); or increasing concentrations of acetylcholine in the presence of 0.02 μM SQ 29548 (closed circles). n is the number of samples measured for each condition tested. In the x-axis, log M [ACh] corresponds to ten fold increases of acetylcholine from 0.001 μM (at −9) to 3 μM (at −6.5). Data are expressed as mean±standard error of the percentage of total relaxation induced by U 46619 . P<0.01 by two-factor ANOVA analysis using StatView software for Apple computers.

### DETAILED DESCRIPTION OF TW INVENTION

As used throughout the disclosure, the following terms, unless otherwise indicated, shall be understood to have the following meanings.

“Patient” refers to animals, preferably mammals, more preferably humans, and includes children and adults.

“Thromboxane inhibitor” refers to any compound that reversibly or irreversibly inhibits thromboxane synthesis, and includes compounds which are the so-called thromboxane A₂ receptor antagonists, thromboxane A₂ antagonists, thromboxane A₂/prostaglandin endoperoxide antagonists, thromboxane receptor (TP) antagonists, thromboxane antagonists, thromboxane synthase inhibitors, and dual acting thromboxane synthase inhibitors and thromboxane receptor antagonists.

“Thromboxane A₂ receptor antagonist” refers to any compound that reversibly or irreversibly blocks the activation of any thromboxane A₂ receptor.

“Thromboxane synthase inhibitor” refers to any compound that reversibly or irreversibly inhibits the enzyme thromboxane synthesis thereby reducing the formation of thromboxane A₂.

“Dual acting thromboxane receptor antagonist and thromboxane synthase inhibitor” refers to any compound that simultaneously acts as a thromboxane A₂ receptor antagonist and a thromboxane synthase inhibitor.

“Therapeutic agent” includes any therapeutic agent that may be used to treat or prevent the diseases or disorders described herein. “Therapeutic agents” include, for example, vasoactive agents, nonsterodial antiinflammmatory compounds (NSAIDs), selective cyclooxygenase-2 (COX-2) inhibitors, anticoagulants, angiotensin converting enzymes (ACE) inhibitors, angiotensin II receptor antagonists, renin inhibitors, and the like. Although NO donors have therapeutic activity, the term “therapeutic agent” does not include the NO donors described herein, since NO donors are separately defined.

“Vasoactive agent” refers to any therapeutic agent capable of relaxing vascular and/or nonvascular smooth muscle. Suitable vasoactive agents include, but are not limited to, potassium channel activators, calcium chanmel blockers, β-blockers, long and short acting α-adrenergic receptor antagonists, prostaglandins, phosphodiesterase inhibitors, adenosine, ergot alkaloids, vasoactive intestinal peptides, dopamine agonists, opioid antagonists, endothelin antagonists, and the like.

“NSAID” refers to a nonsteroidal anti-inflammatory compound or a nonsteroidal anti-inflammatory drug. NSAIDs inhibit cyclooxygenase, the enzyme responsible for the biosyntheses of the prostaglandins and certain autocoid inhibitors, including inhibitors of the various isozymes of cyclooxygenase (including but not limited to cyclooxygenase-1 and -2), and as inhibitors of both cyclooxygenase and lipoxygenase.

“Cyclooxygenase-2 (COX-2) inhibitor” refers to a compound that selectively inhibits the cyclooxygenase-2 enzyme over the cyclooxygenase-1 enzyme. Preferably, the compound has a cyclooxygenase-2 IC₅₀ of less than about 0.5 μM, and also has a selectivity ratio of cyclooxygenase-2 inhibition over cyclooxygenase-1 inhibition of at least 50, and more preferably of at least 100. Even more preferably, the compound has a cyclooxygenase-1 IC₅₀ of greater than about 1 μM, and more preferably of greater than 20 μM. The compound can also inhibit the enzyme, lipoxygenase. Such preferred selectivity may indicate an ability to reduce the incidence of common NSAID-induced side effects.

“Renal disease” refers to any disease, disorder or dysfunction of the kidney of a patient including, for example, chronic glomercular disease, such as for example, glomerulonephritis, lupus nephritis and systemic lupus erythematosus, renal failure, renal hypertension, hepatorenal syndrome, proteinurea, such as, for example, albuminurea and microalbuminurea, haemolytic uraemic syndromes and renal impairment following ureteral obstruction.

“Gastrointestinal disorder” refers to any disease or disorder of the upper gastrointestinal tract of a patient including, for example, peptic ulcers, stress ulcers, gastric hyperacidity, dyspepsia, gastroparesis, Zollinger-Ellison syndrome, gastroesophageal reflux disease, short-bowel (anastomosis) syndrome, hypersecretory states associated with systemic mastocytosis or basophilic leukemia and hyperhistaminemia, and bleeding peptic ulcers that result, for example, from neurosurgery, head injury, severe body trauma or burns.

“Upper gastrointestinal tract” refers to the esophagus, the stomach, the duodenum and the jejunum.

“Ulcers” refers to lesions of the upper gastrointestinal tract lining that are characterized by loss of tissue. Such ulcers include gastric ulcers, duodenal ulcers and gastritis.

“Topical” refers to the delivery of a compound by passage through the skin and into the blood stream and includes transdermal delivery.

“Transmucosal” refers to delivery of a compound by passage of the compound through the mucosal tissue and into the blood stream.

“Penetration enhancement” or “permeation enhancement” refers to an increase in the permeability of the skin or mucosal tissue to a selected pharmacologically active compound such that the rate at which the compound permeates through the skin or mucosal tissue is increased.

“Carriers” or “vehicles” refers to carrier materials suitable for compound administration and include any such material known in the art such as, for example, any liquid, gel, solvent, liquid diluent, solubilizer, or the like, which is non-toxic and which does not interact with any components of the composition in a deleterious manner.

“Nitric oxide adduct” or “NO adduct” refers to compounds and functional groups which, under physiological conditions, can donate, release and/or directly or indirectly transfer any of the three redox forms of nitrogen monoxide (NO⁺, NO⁻, NO.), such that the biological activity of the nitrogen monoxide species is expressed at the intended site of action.

“Nitric oxide releasing” or “nitric oxide donating” refers to methods of donating, releasing and/or directly or indirectly transferring any of the three redox forms of nitrogen monoxide (NO+, NO⁻, NO.), such that the biological activity of the nitrogen monoxide species is expressed at the intended site of action.

“Nitric oxide donor” or “NO donor” refers to compounds that donate, release and/or directly or indirectly transfer a nitrogen monoxide species, and/or stimulate the endogenous production of nitric oxide or endothelium-derived relaxing factor (EDRF) in vivo and/or elevate endogenous levels of nitric oxide or EDRF in vivo. “NO donor” also includes compounds that are substrates for nitric oxide synthase.

“Alkyl” refers to a lower alkyl group, a haloalkyl group, an alkenyl group, an alkynyl group, a bridged cycloalkyl group, a cycloalkyl group or a heterocyclic ring, as defined herein.

“Lower alkyl” refers to branched or straight chain acyclic alkyl group comprising one to about ten carbon atoms (preferably one to about eight carbon atoms, more preferably one to about six carbon atoms). Exemplary lower allyl groups include methyl, ethyl, n-propyl, isopropyl, n-butyl, isobutyl, sec-butyl, t-butyl, pentyl, neopentyl, iso-amyl, hexyl, octyl, and the like.

“Haloalkyl” refers to a lower alkyl group, an alkenyl group, an alkynyl group, a bridged cycloallyl group, a cycloalkyl group or a heterocyclic ring, as defined herein, to which is appended one or more halogens, as defined herein. Exemplary haloalkyl groups include trifluoromethyl, chloromethyl, 2-bromobutyl, 1-bromo-2-chloro-pentyl, and the like.

“Alkenyl” refers to a branched or straight chain C₂-C₁₀ hydrocarbon (preferably a C₂-C₈ hydrocarbon, more preferably a C₂-C₆ hydrocarbon) which can comprise one or more carbon-carbon double bonds. Exemplary alkenyl groups include propylenyl, buten-1-yl, isobutenyl, penten-1-yl, 2,2-methylbuten-1-yl, 3-methylbuten-1-yl, hexan-1-yl, hepten-1-yl, octen-1-yl, and the like.

“Alkynyl” refers to an unsaturated acyclic C₂-C₁₀ hydrocarbon (preferably a C₂-C₈ hydrocarbon, more preferably a C₂-C₆ hydrocarbon) which can comprise one or more carboncarbon triple bonds. Exemplary alkynyl groups include ethynyl, propynyl, butyn-1-yl, butyn-2-yl, pentyl-1-yl, pentyl-2-yl, 3-methylbutyn-1-yl, hexyl-1-yl, hexyl-2-yl, hexyl-3-yl, 3,3-dimethyl-butyn-1-yl, and the like.

“Bridged cycloalkyl” refers to two or more cycloalkyl groups, heterocyclic groups, or a combination thereof fused via adjacent or non-adjacent atoms. Bridged cycloalkyl groups can be unsubstituted or substituted with one, two or three substituents independently selected from alkyl, alkoxy, amino, alkylamino, dialkylamino, hydroxy, halo, carboxyl, alkylcarboxylic acid, aryl, amidyl, ester, alkylcarboxylic ester, carboxamido, alkylcarboxamido, oxo and nitro. Exemplary bridged cycloalkyl groups include adamantyl, decahydronapthyl, quinuclidyl, 2,6-dioxabicyclo[3.3.0]octane, 7-oxabycyclo[2.2.1]heptyl, 8-azabicyclo[3,2,1]oct-2-enyl and the like.

“Cycloalkyl” refers to a saturated or unsaturated cyclic hydrocarbon comprising from about 3 to about 10 carbon atoms. Cycloalkyl groups can be unsubstituted or substituted with one, two or three substituents independently selected from alkyl, alkoxy, amino, alkylamino, dialkylamino, arylamino, diarylamino, alkylarylamino, aryl, amidyl, ester, hydroxy, halo, carboxyl, alkylcarboxylic acid, alkylcarboxylic ester, carboxamido, alkylcarboxamido, oxo and nitro. Exemplary cycloalkyl groups include cyclopropyl, cyclobutyl, cyclopentyl, cyclohexyl, cyclohexenyl, cyclohepta-1,3-dienyl, and the like.

“Heterocyclic ring or group” refers to a saturated, unsaturated, cyclic or aromatic or polycyclic hydrocarbon group having about 2 to about 10 carbon atoms (preferably about 4 to about 6 carbon atoms) where 1 to about 4 carbon atoms are replaced by one or more nitrogen, oxygen and/or sulfur atoms. Sulfur may be in the thio, sulfinyl or sulfonyl oxidation state. The heterocyclic ring or group can be fused to an aromatic hydrocarbon group. Heterocyclic groups can be unsubstituted or substituted with one, two or three substituents independently selected from alkyl, alkoxy, amino, alkylamino, dialkylamino, arylamino, diarylamino, alkylarylamino, hydroxy, oxo, thial, halo, carboxyl, carboxylic ester, alkylcarboxylic acid, allcylcarboxylic ester, aryl, arylcarboxylic acid, arylcarboxylic ester, amidyl, ester, carboxamido, alkylcarboxamido, arylcarboxamido, sulfonic acid, sulfonic ester, sulfonamido and nitro. Exemplary heterocyclic groups include pyrrolyl, 3-pyrrolinyl-4,5,6-trihydro-2H-pyranyl, -pyridinyl, 1,4-dihydropyridinyl, pyrazolyl, triazolyl, pyrimidinyl, pyridazinyl, oxazolyl, thiazolyl, imidazolyl, indolyl, thiophenyl, furanyl, tetrhydrofuranyl, tetrazolyl, 2-pyrrolinyl, 3-pyrrolinyl, pyrrolindinyl, oxazolindinyl 1,3-dioxolanyl, 2,6-dioxabicyclo[3,3,0]octanyl, 2-imidazonlinyl, imnidazolindinyl, 2-pyrazolinyl, pyrazolidinyl, isoxazolyl, isothiazolyl, 1,2,3-oxadiazolyl, 1,2,3-triazolyl, 1,3,4-thiadiazolyl, 2H-pyranyl, 4H-pyranyl, piperidinyl, 1,4-dioxanyl, morpholinyl, 1,4-dithianyl, thiomorpholinyl, pyrazinyl, piperazinyl, 1,3,5-triazinyl, 1,3,5-trithianyl, benzo(b)thiophenyl, benzimidazolyl, quinolinyl, and the like.

“Heterocyclic compounds” refer to mono- and polycyclic compounds comprising at least one aryl or heterocyclic ring.

“Aryl” refers to a monocyclic, bicyclic, carbocyclic or heterocyclic ring system comprising one or two aromatic rings. Exemplary aryl groups include phenyl, pyridyl, napthyl, quinoyl, tetrahydronaphthyl, furanyl, indanyl, indenyl, indoyl, and the like. Aryl groups (including bicylic aryl groups) can be unsubstituted or substituted with one, two or three substituents independently selected from alkyl, alkoxy, amino, alkylamino, dialkylamino, arylamino, diarylamino, alkylarylamino, hydroxy, carboxyl, carboxylic ester, alkylcarboxylic acid, alkylcarboxylic ester, aryl, arylcarboxylic acid, arylcarboxylic ester, alkylcarbonyl, arylcarbonyl, amidyl, ester, carboxamido, alkylcarboxamido, carbomyl, sulfonic acid, sulfonic ester, sulfonamido and nitro. Exemplary substituted aryl groups include tetrafluorophenyl, pentafluorophenyl, sulfonamide, alkylsulfonyl, arylsulfonyl, and the like.

“Alkylaryl” refers to an alkyl group, as defined herein, to which is appended an aryl group, as defined herein. Exemplary alkylaryl groups include benzyl, phenylethyl, hydroxybenzyl, fluorobenzyl, fluorophenylethyl, and the like.

“Arylalkyl” refers to an aryl radical, as defined herein, attached to an alkyl radical, as defined herein.

“Cycloalkylalkyl” refers to a cycloalkyl radical, as defined herein, attached to an alkyl radical, as defined herein.

“Heterocyclicalkyl” refers to a heterocyclic ring radical, as defined herein, attached to an alkyl radical, as defined herein.

“Cycloalkenyl” refers to an unsaturated cyclic hydrocarbon having about 3 to about 10 carbon atoms (preferably about 3 to about 8 carbon atoms, more preferably about 3 to about 6 carbon atoms) comprising one or more carbon-carbon double bonds.

“Arylheterocyclic ring” refers to a bi- or tricyclic ring comprised of an aryl ring, as defined herein, appended via two adjacent carbon atoms of the aryl ring to a heterocyclic ring, as defined herein. Exemplary arylheterocyclic rings include dihydroindole, 1,2,3,4-tetrahydroquinoline, and the like.

“Alkoxy” refers to R₅₀O—, wherein R₅₀ is an alkyl group, as defined herein. Exemplary alkoxy groups include methoxy, ethoxy, t-butoxy, cyclopentyloxy, and the like.

“Arylalkoxy or alkoxyaryl” refers to an alkoxy group, as defined herein, to which is appended an aryl group, as defined herein. Exemplary arylalkoxy groups include benzyloxy, phenylethoxy, chlorophenylethoxy, and the like.

“Aryloxy” refers to R₅₅O—, wherein R₅₅ is an aryl group, as defined herein. Exemplary aryloxy groups include napthyloxy, quinolyloxy, isoquinolizinyloxy, and the like.

“Alkoxyalkyl” refers to an alkoxy group, as defined herein, appended to an alkyl group, as defined herein. Exemplary alkoxyalkyl groups include methoxymethyl, methoxyethyl, isopropoxymethyl, and the like.

“Alkoxyhaloalkyl” refers to an alkoxy group, as defined herein, appended to a haloalkyl group, as defined herein. Exemplary alkoxyhaloalkyl groups include 4-methoxy-2-chlorobutyl and the like.

“Cycloalkoxy” refers to R₅₄O—, wherein R₅₄ is a cycloalkyl group or a bridged cycloalkyl group, as defined herein. Exemplary cycloalkoxy groups include cyclopropyloxy, cyclopentyloxy, cyclohexyloxy, and the like.

“Haloalkoxy” refers to a haloalkyl group, as defined herein, to which is appended an alkoxy group, as defined herein. Exemplary haloalkyl groups include 1,1,1-trichloroethoxy, 2-bromobutoxy, and the like.

“Hydroxy” refers to —OH.

“Oxo” refers to ═O.

“Oxy” refers to —O⁻R₇₇⁺ wherein R₇₇ is an organic or inorganic cation.

“Organic cation” refers to a positively charged organic ion. Exemplary organic cations include alkyl substituted ammonium cations, and the like.

“Inorganic cation” refers to a positively charged metal ion. Exemplary inorganic cations include Group I metal cations such as for example, sodium, potassium, and the like.

“Hydroxyalkyl” refers to a hydroxy group, as defined herein, appended to an alkyl group, as defined herein.

“Amino” refers to —NH₂.

“Nitrate” refers to —O—NO₂.

“Nitrite” refers to —O—NO.

“Thionitrate” refers to —S—NO₂.

“Thionitrite” and “nitrosothiol” refer to —S—NO.

“Nitro” refers to the group —NO₂ and “nitrosated” refers to compounds that have been substituted therewith.

“Nitroso” refers to the group —NO and “nitrosylated” refers to compounds that have been substituted therewith.

“Nitrile” and “cyano” refer to —CN.

“Halogen” or “halo” refers to iodine (I), bromine (Br), chlorine (Cl), and/or fluorine (F).

“Alkylamino” refers to R₅₀NH—, wherein R₅₀ is an alkyl group, as defined herein. Exemplary alkylamino groups include methylamino, ethylamino, butylamino, cyclohexylamino, and the like.

“Arylamino” refers to R₅₅NH—, wherein R₅₅ is an aryl group, as defined herein.

“Dialkylamino” refers to R₅₀R₅₂N—, wherein R₅₀ and R₅₂ are each independently an alkyl group, as defined herein. Exemplary dialkylamino groups include dimethylamino, diethylamino, methyl propargylamino, and the like.

“Diarylamino” refers to R₅₅R₆₀N—, wherein R₅₅ and R₆₀ are each independently an aryl group, as defined herein.

“Alkylarylamino” refers to R₅₀R₅₅N—, wherein R₅₀ is an alkyl group, as defined herein, and R₅₅ is an aryl group, as defined herein.

“Aminoalkyl” refers to an amino group, an alkylamino group, a dialkylamino group, an arylamino group, a diarylamino group, an alkylarylamino group or a heterocyclic ring, as defined herein, to which is appended an allyl group, as defined herein.

“Aminoaryl” refers to an amino group, an alkylamino group, a dialkylamino group, an arylamino group, a diarylamino group, an alkylarylamino group or a heterocyclic ring, as defined herein, to which is appended an aryl group, as defined herein.

“Thio” refers to —S—.

“Sulfinyl” refers to —S(O)—.

“Methanthial” refers to —C(S)—.

“Thial” refers to ═S.

“Sulfonyl” refers to —S(O)₂⁻.

“Sulfonic acid” refers to —S(O)₂OR₇₆, wherein R₇₆ is a hydrogen, an organic cation or an inorganic cation.

“Alkylsulfonic acid” refers to a sulfonic acid group, as defined herein, appended to an alkyl group, as defined herein.

“Arylsulfonic acid” refers to an sulfonic acid group, as defined herein, appended to an aryl group, as defined herein

“Sulfonic ester” refers to —S(O)₂OR₅₈, wherein R₅₈ is an alkyl group, an aryl group, an alkylaryl group or an aryl heterocyclic ring, as defined herein.

“Sulfonamido” refers to —S(O)₂—N(R₅₁)(R₅₇), wherein R₅₁ and R₅₇ are each independently a hydrogen atom, an alkyl group, an aryl group, an alkylaryl group, or an arylheterocyclic ring, as defined herein, or R₅₁ and R₅₇ taken together are a heterocyclic ring, a cycloalkyl group or a bridged cycloalkyl group, as defined herein.

“Alkylsulfonamido” refers to a sulfonamido group, as defined herein, appended to an alkyl group, as defined herein.

“Arylsulfonamido” refers to a sulfonamido group, as defined herein, appended to an aryl group, as defined herein.

“Alkylthio” refers to R₅₀S—, wherein R₅₀ is an alkyl group, as defined herein.

“Arylthio” refers to R₅₅S—, wherein R₅₅ is an aryl group, as defined herein.

“Cycloalkylthio” refers to R₅₄S—, wherein R₅₄ is a cycloalkyl group or a bridged cycloalkyl group, as defined herein. Exemplary cycloalkylthio groups include cyclopropylthio, cyclopentylthio, cyclohexylthio, and the like.

“Alkylsulfinyl” refers to R₅₀—S(O)—, wherein R₅₀ is an alkyl group, as defined herein.

“Alkylsulfonyl” refers to R₅₀—S(O)₂—, wherein R₅₀ is an alkyl group, as defined herein.

“Arylsulfinyl” refers to R₅₅—S(O)—, wherein R₅₅ is an aryl group, as defined herein.

“Arylsulfonyl” refers to R₅₅—S(O)₂—, wherein R₅₅ is an aryl group, as defined herein.

“Amidyl” refers to R₅₁C(O)N(R₅₇)— wherein R₅₁ and R₅₇ are each independently a hydrogen atom, an alkyl group, an aryl group, an alkylaryl group, or an arylheterocyclic ring, as defined herein.

“Ester” refers to R₅₁C(O)O— wherein R₅₁ is a hydrogen atom, an alkyl group, an aryl group, an alkylaryl group, or an arylheterocyclic ring, as defined herein.

“Carbamoyl” refers to —O—C(O)N(R₅₁)(R₅₇), wherein R₅₁, and R₅₇ are each independently a hydrogen atom, an alkyl group, an aryl group, an alkylaryl group or an arylheterocyclic ring, as defined herein, or R₅₁ and R₅₇ taken together are a heterocyclic ring, a cycloalkyl group or a bridged cycloalkyl group, as defined herein.

“Carbamate” refers to —R₅₁O—C(O)N(R₅₇), wherein R₅₁ and R₅₇ are each independently a hydrogen atom, an alkyl group, an aryl group, an alkylaryl group or an arylheterocyclic ring, as defined herein, or R₅₁ and R₅₇ taken together are a heterocyclic ring, a cycloalkyl group or a bridged cycloalkyl group, as defined herein.

“Carboxyl” refers to —C(O)OR₇₆, wherein R₇₆ is a hydrogen, an organic cation or an inorganic cation, as defined herein.

“Carbonyl” refers to —C(O)—.

“Alkylcarbonyl” or “alkanoyl” refers to R₅₀—C(O)—, wherein R₅₀ is an alkyl group, as defined herein.

“Arylcarbonyl” or “aroyl” refers to R₅₅—C(O)—, wherein R₅₅ is an aryl group, as defined herein.

“Carboxylic ester” refers to —C(O)OR₅₈, wherein R₅₈ is an alkyl group, an aryl group, an alkylaryl group or an aryl heterocyclic ring, as defined herein.

“Alkylcarboxylic acid” and “alkylcarboxyl” refer to an alkyl group, as defined herein, appended to a carboxyl group, as defined herein.

“Alkylcarboxylic ester” refers to an alkyl group, as defined herein, appended to a carboxylic ester group, as defined herein.

“Arylcarboxylic acid” refers to an aryl group, as defined herein, appended to a carboxyl group, as defined herein.

“Arylcarboxylic ester” and “arylcarboxyl” refer to an aryl group, as defined herein, appended to a carboxylic ester group, as defined herein.

“Carboxamido” refers to —C(O)N(R₅₁)(R₅₇), wherein R₅₁ and R₅₇ are each independently a hydrogen atom, an alkyl group, an aryl group, an alkylaryl group or an arylheterocyclic ring, as defined herein, or R₅₁ and R₅₇ taken together with the nitrogen to which they are attached form a heterocyclic ring, a cycloalkyl group or a bridged cycloalkyl group, as defined herein.

“Alkylcarboxamido” refers to an alkyl group, as defined herein, appended to a carboxamido group, as defined herein.

“Arylcarboxamido” refers to an aryl group, as defined herein, appended to a carboxamido group, as defined herein.

“Urea” refers to —N(R₅₉)—C(O)N(R₅₁)(R₅₇) wherein R₅₁, R₅₇, and R₅₉ are each independently a hydrogen atom, an alkyl group, an aryl group, an alkylaryl group, or an arylheterocyclic ring, as defined herein, or R₅₁ and R₅₇ taken together with the nitrogen to which they are attached form a heterocyclic ring, as defined herein.

“Phosphoryl” refers to —P(R₇₀)(R₇₁)(R₇₂), wherein R₇₀ is a lone pair of electrons, sulfur or oxygen, and R₇₁ and R₇₂ are each independently a covalent bond, a hydrogen, a lower alkyl, an alkoxy, an alkylamino, a hydroxy or an aryl, as defined herein.

“Silyl” refers to —Si(R₇₃)(R₇₄)(R₇₅), wherein R₇₃, R₇₄ and R₇₅ are each independently a covalent bond, a lower alkyl, an alkoxy, an aryl or an arylalkoxy, as defined herein.

The term “sexual dysfunction” generally includes any sexual dysfunction in a patient, including an animal, preferably a mammal, more preferably a human. The patient may be male or female and may preferably be diabetic. Sexual dysfunctions can include, for example, sexual desire disorders, sexual arousal disorders, orgasmic disorders and sexual pain disorders. Female sexual dysfunction refers to any female sexual dysfunction including, for example, sexual desire disorders, sexual arousal dysfunctions, orgasmic dysfunctions, sexual pain disorders, dyspareunia, and vaginismus. The female may be pre-menopausal or menopausal. Sexual dysfunction may be caused, for example, by pregnancy, menopause, cancer, pelvic surgery, chronic medical illness or medications. Male sexual dysfunction refers to any male sexual dysfunctions including, for example, male erectile dysfunction and impotence.

The present invention is directed to the treatment and/or prevention of sexual dysfunctions in patients, including males and females, by administering the compounds and compositions described herein. The present invention is also directed to improving and/or enhancing sexual responses in patients, including males and females, by administering the compounds and/or compositions described herein. The patient may preferably be diabetic. The novel compounds and novel compositions of the present invention are described in more detail herein.

Contemplated thromboxane inhibitors for use in the present invention include, for example, 2-(acetyloxy)-benzoic acid, AA 2414 (seratrodast, (4-hydroxy-(Z)-(2,4,5-trimethyl-3,6-dioxo-1,4-cyclohexadien-1-yl)-benezeneheptanoic acid), AH 2,848 ((1α(Z), 2β,5α)-(±)-7-(5-(((1,1′-biphenyl)-4-yl)methoxy)-2-(4-morpholinyl)-3-oxocyclopentyl)-4-heptenoic acid), BAY u3405 (ramatroban, 3R-((4-fluorophenyl)sulfonyl)amino)-1,2,3,4-tetrahydro-9H-carbazole-9-propanoic acid), BM 13177 (sulotroban, ((4-(2-((phenylsulfonyl)amino)ethyl)phenoxy)-acetic acid), BM 13505 (daltroban, (4-(2-(((4-chlorophenyl)sulfonyl)amino)ethyl)-benzeneacetic acid), BMS 180291 (ifetroban, ((+)-1S-(1α,2α,3α,4α)-2-((3-(4-(n-pentylamino)carbonyl)-2-oxazolyl)-7-oxabicyclo)(2.2.1)hept-2-yl)methyl)benzene proponic acid), cinnamorphilin ((8R,8′S)-4,4′-dihydroxy-3,3′-dimethoxy-7-oxo-8,8′-neolignan), CGS 12970 (3-methyl-2-(3-pyridyl)indole-1-octanoic acid), CGS 15435, CGS 22652 (4-(((4-chlorophenyl)sulfonyl)amino)butyl)-3-pyridineheptanoic acid), CV 4151 ((E)-7-phenyl-7-(3-pyridinyl)-6-heptenoic acid), dazoxiben (4-(2-(1H-imidazol-1-yl)ethoxy)benzoic acid), dazmegrel, DT-TX 30 ((E)-6-(4-2-(4-chlorobenzene sulphonylamino)ethyl)phenyl)-6-(3-pyridyl)-hex-5-enoic acid), etofibrate (2-(2-(4-chlorophenoxy)-2-methyl-1-oxopropoxy)-3-pyridinecarboxylic acid ethyl ester), EP 045 ((1α,2β(Z),3α,4α)-7-(3-((((phenylamino)carbonyl)hydrazono)methyl)bicyclo(2.2.1) hept-2-yl), EP 092 (7-((1S,2S,3S,4R)-3-(1-(3-(phenylthioureidoimino)ethyl)-bicyclo(2.2.1)heptane-2-yl)-5-heptenoic acid), F 10171 (1-(((5-(4-chlorophenyl)-2-furanyl)methylene)amino)-3-(4-(1-piperazinyl)butyl)-2,4-imidazolidinedione), FCE 27262 ((E)-(±)-5-(((1-cyclohexyl-2-(1H-imidazol-1-yl)-3-phenylpropylidene)amino)oxy-pentanoic acid), FI 2845 (camongrel, 2,3-dihydro-5-(2-(1H-imidazol-1-yl)ethoxy)-indene-1-carboxylic acid), FK 070 ((5Z)-6-((2S,4R)-4-((4-chlorophenyl)sulfonyl)amino)-1-(3-pyridinylmethyl)-2-pyrrolidinyl)-5-hexenoic acid monohydrate), furegrelate, GR 32191 (vapiprost, (1R-(1α(Z),2β,3β,5α))-(+)-7-(5-((1,1′-biphenyl)-4-ylmethoxy)-3-hydroxy-2-(1-piperidinyl)cyclopentyl)-4-++++heptonoic acid), GR 83783 ((±)-(1α(Z),2β,5β)-6-((2-(hexahydro-1H-azepin-1yl)-5-((4-(4-propyl-3-pyridinyl)phenyl)methoxy)cyclopentyl)oxy)-4-hexanoic acid), GR 85305 ((5E)-6-(3-(2-((4-iodophenyl)sulfonyl)amino)ethyl)phenyl)-6-(3-pyridinyl)-5-hexenoic acid), GR 108774 ((5E)-6-(3-(2-((4-iodophenyl)sulfonyl)amnino)-1,1-dimethylethyl)phenyl)-6-(3-pyridinyl)-5-hexenoic acid), IBI P-05006 (2-(6-carboxyhexyl)-3-n-hexylcyclohexyl amine), isbogrel ((E)-7-phenyl-7-(3-pyridinyl)-6-heptenoic acid), ICI 180080 (5(Z)-7-(2,2-dimethyl-4-(2-hydroxyphenyl)-1,3-dioxan-cis-5-yl)heptenoic acid), ICI 192605 ((Z)-(2α,4α5α)-6-(2-(2-chlorophenyl)-4-(2-hydroxyphenyl)-1,3-dioxan-5-yl)-4-hexenoic acid), KT 2962 (3-(4-(((4-chlorophenyl)sulfonyl)amino)butyl)-6-(1-methylethyl)-1-azulenesulfonic acid monosodium salt), KY 234 ((E)-11-(2-(5,6-dimethyl-1H-benzimidazol-1-yl)-6,11-dihydro-dibenz(b,e) oxepin-2-carboxylic acid sodium salt), KW 3635 (sodium (E)-11-(2-(5,6-dimethyl-1-benzimidazolyl)ethylidene)-6,11-dihydrodibenz (b,e)oxepin-2-carboxylate monohydrate), linotroban (4-methyl-benzenesulfonate-N-((phenylmethoxy)carbonyl)-serine ethyl ester), L 640035 (dibenzo(b,f)thiepin-3-methanol-5,5-dioxide), L 670596 ((−)-6,8-difloro-2,3,4,9-tetrahydro-9-((4-(methylsulfonyl)phenyl)methyl)-1H-carbazole-1-acetic acid), L 636499 ((5,5-dioxide-dibenzo(b,f)thiepin-3-carboxylic acid), L 655240 (3-methyl-1-((4-chlorophenyl)methyl)-5-fluoro-α,α,-1H-indole-2-propanoic acid), midazogrel, ON 579 ((4-((2-(((4-chlorophenyl)sulfonyl)amino)ethyl)thio)-2,6-difluoro phenoxy)acetic acid), ONO 3708 ((1S-(1α,2β(Z),3α(S),5α))-7-(3-((cyclopentyl hydroxyacetyl)amino)-6,6-dimethylbicyclo (3.1.1hept-2-yl)-5-heptenoic acid), OKY 046 (ozagrel hydrochloride, (E)-3-(4-(1H-imidazol-1-ylmethyl)phenyl)-2-propenoic acid monohydrochloride), OKY 1555, OKY 1580, OKY 1581, (E)-2-methyl-3-(4-(3-pyridinylmethyl)phenyl)-2-propenoic acid sodium salt), S-145 ((±)-(5Z)-7-(3-endo-((phenylsulfonyl)amino)bicyclo(2.2.1)hept-2-exo-yl)heptenoic acid), picotamide (4-methoxy-N-N′-bis(3-pyridinylmethyl)-1,3-benzenedicarboxamide monohydrate), R 68070 (ridogrel, (E)-5-(((3-pyridinyl(3-(trifluoromethyl)phenyl)methylene)amino)oxy)-pentanoic acid), S-1452 (domitroban, (1R)-(1α2α(Z),3β,4α))-7-(3-((phenylsulfonyl)amino)bicyclo (2.2.1)hept-2-yl)-5-heptenoic acid), SKF 88046 (N7-(3-chlorophenyl)-N2-((7-(((3-chlorophenyl)amino)sulfonyl)-3,4-dihydro-2(1H)-isoquinolinyl)sulfonyl)-3,4-dihydro-2,7(1H)-isoquinol disulfonamide), SQ 27427 (((1S)-(1α,2α(Z),3α(1E,3R),4α))-7-(3-(3-cyclohexyl-3-hydroxy-1-propenyl)-7-oxabicyclo(2.2.1)hept-2-yl)-5-heptenoic acid), SQ 28668 ((1α,2α(Z), 3α(1E,3S,4R),4α))-7-(3-(3-hydroxy-4-phenyl-1-pentyl)-7-oxabicyclo(2.2.1)hept-2-yl)-5-heptenoic acid), SQ 29548 (((1S)-(1α,2β(5Z),3β,4α)-7-(3-((2-((phenylamino)-carbonyl)hydrazino)methyl)-7-oxabicyclo(2.2.1)-hept-2-yl)-5-heptenoic acid), SQ 30741 (((1S)-(1α,2α(Z),3α,4α))-7-(3-(((((1-oxoheptyl)amino)acetyl)amino)methy)-7-oxabicyclo(2.2.1)-hept-2-yl)-5-heptenoic acid), SQ 33961 ((1-exo,exo))-2-((3-(4-(((4-cyclohexylbutyl)amino)carbonyl)-2-oxazolyl)-7-oxabicyclo(2.2.1)-hept-2-yl)methyl)-benzenepropanoic acid), TER 930180 (4-(1-(((4-chlorophenyl)sulfonyl)amino)methyl)-4-(3-pyridinyl)butyl)-benzenepropanoic acid), UK 34787, UP 11677 (mipitroban (β,β-dimethyl-6-chloro-3-((4-chlorophenyl)methyl)-3H-imidazo(4,5-b)pyridine-2-butanoic acid), Y 20811, YM 158 ((3-((4-tert-butylthiazol-2-yl)methoxy)-5′-(3(4-chlorobenzenesulfonyl)propyl-2′-(1-tetrazol-5-ylmethoxy) benzanilide monosodium salt monohydrate), Z 335 ((±)-sodium(2-(4-(chloro phenylsulfonyl aminomethyl)indan-5-yl)acetate monohydrate), ZD 1542 (4(Z)-6-(2S,4S,5R)-2-(1methyl-1-(2-nitro-4-tolyloxy)ethyl)-4-(3-pyridyl)-1,3-dioxan-5-yl)hex-4-acid), ZD 9583 ((4Z)-6-((2S,4S,5R)-2-(1-(2-cyano-4-methylphenoxy)-1-methylethyl)-4-(3-pyridyl)-1,3-dioxan-5-yl)hex-4-enoicn acid).

Although the above compounds are collectively referred to as “thromboxane inhibitors”, one skilled in the art will readily recognize whether any particular compound is specifically a thromboxane A₂ receptor antagonist, a thromboxane synthase inhibitor or a dual-acting thromboxane receptor antagonist and thromboxane synthase inhibitor.

Sources of information for the above compounds include Goodman and Gilman, The Pharmacological Basis of Therapeutics (9th Ed.), McGraw-Hill, Inc. (1996), The Physician's Desk Reference (49th Ed.), Medical Economics (1995), Drug Facts and Comparisons (1993 Ed), Facts and Comparisons (1993), Merck Index on CD-ROM, Twelfth Edition, Version 12:1, (1996), STN Express, file phar and file registry, the disclosures of each of which are incorporated herein by reference in their entirety.

A principal aspect of the present invention relates to novel compositions comprising at least one thromboxane inhibitor, nitric oxide and/or at least one compound that donates, transfers or releases nitric oxide and/or stimulates endogenous production of nitric oxide or EDRF in vivo and/or is a substrate for nitric oxide synthase, and/or otherwise directly or indirectly deliver or transfer nitric oxide to a site of its activity, such as on a cell membrane, in vivo.

As used herein, the term “nitric oxide” encompasses uncharged nitric oxide (NO.) and charged nitrogen monoxide species, preferably charged nitrogen monoxide species, such as nitrosonium ion (NO⁺) and nitroxyl ion (NO−). NO. is a highly reactive short-lived species that is potentially toxic to cells. This is critical because the pharmacological efficacy of NO depends upon the form in which it is delivered. In contrast to the nitric oxide radical (NO.), nitrosonium (NO⁺) does not react with O₂ or O₂⁻ species, and functionalities capable of transferring and/or releasing NO⁺ and NO− are also resistant to decomposition in the presence of many redox metals.

Compounds contemplated for use in the present invention (e.g., thromboxane inhibitors) may be used in combination with nitric oxide and compounds that release nitric oxide (i.e., compounds that release nitric oxide or otherwise directly or indirectly deliver or transfer nitric oxide to a site of its activity, such as on a cell membrane, and/or elevate or stimulate production of endogenous nitric oxide or EDRF in vivo and/or is a substrate for nitric oxide synthase.

The term “nitric oxide” encompasses uncharged nitric oxide (NO.) and charged nitrogen monoxide species, preferably charged nitrogen monoxide species, such as nitrosonium ion (NO⁺) and nitroxyl ion (NO−). The reactive form of nitric oxide may be provided by gaseous nitric oxide. The nitrogen monoxide releasing, delivering or transferring compounds include any and all such compounds which provide nitrogen monoxide to its intended site of action in a form active for its intended purpose. The term “NO adducts” encompasses any nitrogen monoxide releasing, delivering or transferring compounds, including, for example, S-nitrosothiols, nitrites, nitrates, S-nitrothiols, sydnonimines, 2-hydroxy-2-nitrosohydrazines (NONOates), (E)-alkyl-2-((E)-hydroxyimino)-5-nitro-3-hexene amines or amides, nitrosoamines, furoxans as well as substrates for the endogenous enzymes which synthesize nitric oxide. NONOates include, but are not limited to, (Z)-1-{N-methy-N-(6-(N-methyl-ammoniohexyl)amino)}diazen-1-ium-1,2-diolate(“MAHMA/NO”), (Z)-1-(N-(3-ammoniopropyl)-N-(n-propyl)amino)diazen-1-ium-1,2-diolate (“PAPA/NO”), (Z)-1-{N-(3-aminopropyl)-N-(4-(3-aminopropylammonio)butyl)-amino}diazen-1-ium-1,2-diolate (spermine NONOate or “SPER/NO”) and sodium (Z)-1-(N,N-diethylamino)diazenium-1,2-diolate(diethylamine NONOate or “DEA/NO”) and derivatives thereof. The “NO adducts” may be mono-nitrosylated, poly-nitrosylated, mono-nitrosated and/or poly-nitrosated or a combination thereof at a variety of naturally susceptible or artificially provided binding sites for biologically active forms of nitrogen monoxide.

One group of NO adducts is the S-nitrosothiols, which are compounds that include at least one —S—NO group., These compounds include S-nitroso-polypeptides (the term “polypeptide” includes proteins and polyamino acids that do not possess an ascertained biological function, and derivatives thereof); S-nitrosylated amino acids (including natural and synthetic amino acids and their stereoisomers and racemic mixtures and derivatives thereof); S-nitrosylated sugars; S-nitrosylated, modified and unmodified, oligonucleotides (preferably of at least 5, and more preferably 5-200 nucleotides); straight or branched, saturated or unsaturated, aliphatic or aromatic, substituted or unsubstituted S-nitrosylated hydrocarbons; and S-nitroso heterocyclic compounds. S-nitrosothiols and methods for preparing them are described in U.S. Pat. Nos. 5,380,758 and 5,703,073; WO 97/27749; WO 98/19672; and Oae et al, Org. Prep. Proc. Int., 15(3):165-198 (1983), the disclosures of each of which are incorporated by reference herein in their entirety.

Another embodiment of the present invention is S-nitroso amino acids where the nitroso group is linked to a sulfur group of a sulfur-containing amino acid or derivative thereof. Such compounds include, for example, S-nitroso-N-acetylcysteine, S-nitroso-captopril, S-nitroso-N-acetylpenicillamine, S-nitroso-homocysteine, S-nitroso-cysteine and S-nitroso-glutathione.

Suitable S-nitrosylated proteins include thiol-containing proteins (where the NO group is attached to one or more sulfur groups on an amino acid or amino acid derivative thereof) from various functional classes including enzymes, such as tissue-type plasminogen activator (TPA) and cathepsin B; transport proteins, such as lipoproteins; heme proteins, such as hemoglobin and serum albumin; and biologically protective proteins, such as immunoglobulin, antibodies and cytokines. Such nitrosylated proteins are described in WO 93/09806, the disclosure of which is incorporated by reference herein in its entirety. Examples include polynitrosylated albumin where one or more thiol or other nucleophilic centers in the protein are modified.

Other examples of suitable S-nitrosothiols include:

(i) HS(C(Rₑ)(R_{f}))ₘSNO;

(ii) ONS(C(Rₑ)(R_{f})ₘRₑ; and

(iii) H₂N—CH(CO₂H)—(CH₂)ₘ—(O)NH—CH(CH₂SNO)—C(O)NH—CH₂—CO₂; H;

wherein m is an integer from 2 to 20; Rₑ and R_{f} are each independently a hydrogen, an alkyl, a cycloalkoxy, a halogen, a hydroxy, an hydroxyalkyl, an alkoxyalkyl, an arylheterocyclic ring, an alkylaryl, a cycloalkylalkyl, a heterocyclicalkyl, an alkoxy, a haloalkoxy, an amino, an alkylamino, a dialkylamino, an arylamino, a diarylamino, an alkylarylamino, an alkoxyhaloalkyl, a haloalkoxy, a sulfonic acid, a sulfonic ester, an alkylsulfonic acid, an arylsulfonic acid, an arylalkoxy, an alkylthio, an arylthio, a cycloalkylthio, a cycloalkenyl, a cyano, an aminoalkyl, an aminoaryl, an aryl, an arylalkyl, an alkylaryl, a carboxarnido, a alkylcarboxamido, an arylcarboxamido, an amidyl, a carboxyl, a carbamoyl, a carbamate, an alkylcarboxylic acid, an arylcarboxylic acid, an alkylcarbonyl, an arylcarbonyl, an ester, a carboxylic ester, an alkylcarboxylic ester, an arylcarboxylic ester, a haloalkoxy, a sulfonamido, an allylsulfonamido, an arylsulfonamido, a sulfonic ester, a urea, a phosphoryl, a nitro, —T—Q , or —(C(Rₑ)(R_{f}))ₖ—T—Q, or Rₑ and R_{f} taken together with the carbons to which they are attached form a carbonyl, a methanthial, a heterocyclic ring, a cycloalkyl group or a bridged cycloalkyl group; Q is —NO or —NO₂; and T is independently a covalent bond, a carbonyl, an oxygen, —S(O)ₒ— or —N(Rₐ)Rᵢ—, wherein o is an integer from 0 to 2, Rₐ is a lone pair of electrons, a hydrogen or an alkyl group; Rᵢ is a hydrogen, an alkyl, an aryl, an alkylcarboxylic acid, an, aryl carboxylic acid, an alkylcarboxylic ester, an arylcarboxylic ester, an alkylcarboxamido, an arylcarboxamido, an alkylaryl, an alkylsulfinyl, an alkylsulfonyl, an arylsulfinyl, an arylsulfonyl, a sulfonamido, a carboxamido, a carboxylic ester, an amino alkyl, an amino aryl, —CH₂—C(T—Q)(Rₑ)(R_{f}), or —(N₂O₂—)⁻.M⁺, wherein M⁺ is an organic or inorganic cation; with the proviso that when Rᵢ is —CH₂—C(T—Q)(Rₑ)(R_{f}) or —(N₂O₂—).M⁺; then “—T—Q” may be a hydrogen, an alkyl group, an alkoxyalkyl group, an aminoalkyl group, a hydroxy group or an aryl group.

In cases where Rₑ and R_{f} are a heterocyclic ring or Rₑ and R_{f} when taken together with the carbon atoms to which they are attached are a heterocyclic ring, then Rᵢ can be a substituent on any disubstituted nitrogen contained within the radical wherein Rᵢ is as defined herein.

Nitrosothiols can be prepared by various methods of synthesis. In general, the thiol precursor is prepared first, then converted to the S-nitrosothiol derivative by nitrosation of the thiol group with NaNO₂ under acidic conditions (pH is about 2.5) which yields the S-nitroso derivative. Acids which may be used for this purpose include aqueous sulfuric, acetic and hydrochloric acids. The thiol precursor can also be nitrosylated by reaction with an organic nitrite such as tert-butyl nitrite, or a nitrosonium salt such as nitrosonium tetraflurorborate in an inert solvent.

Another group of NO adducts for use in the present invention, where the NO adduct is a compound that donates, transfers or releases nitric oxide, include compounds comprising at least one ON—O—, ON—N— or ON—C— group. The compounds that include at least one ON—O—, ON—N— or ON—C— group are preferably ON—O—, ON—N— or ON—C-polypeptides (the term “polypeptide” includes proteins and polyamino acids that do not possess an ascertained biological function, and derivatives thereof); ON—O, ON—N— or ON—C-amino acids (including natural and synthetic amino acids and their stereoisomers and racemic mixtures); ON—O—, ON—N— or ON—C-sugars; ON—O—, ON—N— or ON—C— modified or unmodified oligonucleotides (comprising at least 5 nucleotides, preferably 5-200 nucleotides); ON—O—, ON—N— or ON-C-straight or branched, saturated or unsaturated, aliphatic or aromatic, substituted or unsubstituted hydrocarbons; and ON—O—, ON—N— or ON—C-heterocyclic compounds.

Another group of NO adducts for use in the present invention include nitrates that donate, transfer or release nitric oxide, such as compounds comprising at least one O₂N—O—, O₂N—N—, O₂N—S— or O₂N—C— group. Preferred among these compounds are O₂N—O—, O₂N—N—, O₂N—S— or O₂N—C— polypeptides (the term “polypeptide” includes proteins and also polyamino acids that do not possess an ascertained biological function, and derivatives thereof); O₂N—O—, O₂N—N—, O₂N—S— or O₂N—C— amino acids (including natural and synthetic amino acids and their stereoisomers and racemic mixtures); O₂N—O—, O₂N—N—, O₂N—S— or O₂N-C-sugars; O₂N—O—, O₂N—N—, O₂N—S— or O₂N—C— modified and unmodified oligonucleotides (comprising at least 5 nucleotides, preferably 5-200 nucleotides); O₂N—O—, O₂N—N—, O₂N—S— or O₂N—C— straight or branched, saturated or unsaturated, aliphatic or aromatic, substituted or unsubstituted hydrocarbons; and O₂N—O—, O₂N—N—, O₂N—S— or O₂N—C— heterocyclic compounds. Preferred examples of compounds comprising at least one O₂N—O—, O₂N—N—, O₂N—S— or O₂N—C— group include isosorbide dinitrate, isosorbide mononitrate, clonitrate, erythrityltetranitrate, mannitol hexanitrate, nitroglycerin, pentaerythritoltetranitrate, pentrinitrol and propatylnitrate.

Another group of NO adducts are N-oxo-N-nitrosoamines that donate, transfer or release nitric oxide and are represented by the formula: R¹R²—N(O—M⁺)—NO, where R¹ and R² are each independently a polypeptide, an amino acid, a sugar, a modified or unmodified oligonucleotide, a straight or branched, saturated or unsaturated, aliphatic or aromatic, substituted or unsubstituted hydrocarbon, or a heterocyclic group, and M⁺ is as defined herein.

Another group of NO adducts are thionitrates that donate, transfer or release nitric oxide and are represented by the formula: R¹—(S)—NO₂, where R¹ is a polypeptide, an amino acid, a sugar, a modified or unmodified oligonucleotide, a straight or branched, saturated or unsaturated, aliphatic or aromatic, substituted or unsubstituted hydrocarbon, or a heterocyclic group. Preferred are those compounds where R¹ is a polypeptide or hydrocarbon with a pair or pairs of thiols that are sufficiently structurally proximate, i.e., vicinal, that the pair of thiols will be reduced to a disulfide. Compounds which form disulfide species release nitroxyl ion (NO−) and uncharged nitric oxide (NO.).

The present invention is also directed to compounds that stimulate endogenous NO or elevate levels of endogenous endothelium-derived relaxing factor (EDRF) in vivo or are substrates for the enzyme, nitric oxide synthase. Such compounds include, for example, L-arginine, L-homoarginine, and N-hydroxy-L-arginine, including their nitrosated and nitrosylated analogs (e.g., nitrosated L-arginine, nitrosylated L-arginine, nitrosated N-hydroxy-L-arginine, nitrosylated N-hydroxy-L-arginine, nitrosated L-homoarginine and nitrosylated L-homoarginine), precursors of L-arginine and/or physiologically acceptable salts thereof, including, for example, citrulline, ornithine, glutamine, lysine, polypeptides comprising at least one of these amino acids, inhibitors of the enzyme arginase (e.g., N-hydroxy-L-arginine and 2(S)-amino-6-boronohexanoic acid) and the substrates for nitric oxide synthase, cytokines, adenosin, bradykinin, calreticulin, bisacodyl, and phenolphthalein. EDRF is a vascular relaxing factor secreted by the endothelium, and has been identified as nitric oxide (NO) or a closely related derivative thereof (Palmer et al, Nature, 327:524-526 (1987); Ignarro et al, Proc. Natl. Acad. Sci. USA, 84:9265-9269 (1987)).

The present invention is also based on the discovery that the administration of at least one thromboxane inhibitor, at least one vasoactive agent and/or at least one nitric oxide donor described herein, are effective for treating or preventing sexual dysfunctions or enhancing sexual responses in patients, including males and females. The patient may preferably be diabetic. The thromboxane inhibitors includes any of those described in the prior art, including those described in the patents, applications and publications cited herein. For example, the patient may be administered a therapeutically effective amount of at least one thromboxane inhibitor, and at least one compound that donates, transfers or releases nitric oxide, elevates endogenous levels of endothelium-derived relaxing factor, or EDRF in vivo, stimulates endogenous synthesis of nitric oxide or is a substrate for nitric oxide synthase. In yet another embodiment, the patient may be administered a therapeutically effective amount of at least one thromboxane inhibitor, and at least one vasoactive agent, and, optionally, at least one compound that donates, transfers or releases nitric oxide, elevates endogenous levels of endothelium-derived relaxing factor, or EDRF in vivo, stimulates endogenous synthesis of nitric oxide or is a substrate for nitric oxide synthase. The compounds may be administered separately or as components of the same composition.

Suitable vasoactive agents include, but are not limited to, potassium channel activators (such as, for example, nicorandil, pinacidil, cromakalim, minoxidil, aprifkalim, loprazolam and the like); calcium channel blockers (such as, for example, nifedipine, veraparmil, diltiazem, gallopamil, niludipine, nimodipins, nicardipine, and the like); βblockers (such as, for example, butixamine, dichloroisoproterenol, propanolol, alprenolol, bunolol, nadolol, oxprenolol, perbutolol, pinodolol, sotalol, timolol, metoprolol, atenolol, acebutolol, bevantolol, pafenolol, tolamodol, and the like); long and short acting a-adrenergic receptor antagonists (such as, for example, phenoxybenzamide, dibenamine, doxazosin, terazosin, phentolamine, tolazoline, prozosin, trimazosin, yohimbine, moxisylyte and the like); prostaglandins (such as, for example, PGE₁, PGA₁, PGB₁, PGF₁, PGF₂, 19-hydroxy-PGA₁, 19-hydroxy-PGB₁, PGE₂, PGA₂, PGB₂, prostacyclins, thromboxanes, leulcotrienes, 6-keto-PGE₁ derivatives and carbacyclin derivatives, and the like); phosphodiesterase inhibitors (such as, for example, papaverine, zaprinast, sildenafil, IC 351); adenosine, ergot alkaloids (such as, for example, ergotatine, ergotamine analogs, including, for example, acetergamine, brazergoline, bromerguride, cianergoline, delorgotrile, disulergine, ergonovine maleate, ergotamine tartrate, etisulergine, lergotrile, lysergide, mesulergine, metergoline, metergotamine, nicergoline, pergolide, propisergide, proterguride, terguride and the like); vasoactive intestinal peptides (such as, for example, peptide histidine isoleucine, peptide histidine methionine, substance P, calcitonin gene-related peptide, neurokinin A, bradykinin, neurokinin B, and the like); doparnine agonists (such as, for example, apomorphine, bromocriptine, testosterone, cocaine, strychnine, and the like); opioid antagonists (such as, for example, naltrexone, and the like);

endothelin antagonists (such as, for example, bosentan, sulfonamide endothelin antagonists, BQ-123, SQ 28608, and the like) and mixtures thereof.

Preferred compositions comprise at least one thromboxane inhibitor (preferably SQ 9548 (((1S)-(1α,2β(5Z),3β,4α))-7-(3-((2-((phenylamino)-carbonyl)hydrazino)methyl)-7-oxabicyclo(2.2.1)-hept-2-yl)-5-heptenoic acid), BAY u3405 (ramatroban, 3R-((4-fluorophenyl)sulfonyl)amino)-1,2,3,4-tetrahydro-9H-carbazole-9-propanoic acid), GR 32191 (vapiprost, (1R-(1α(Z),2β,3β,5α))-(+)-7-(5-((1,1′-biphenyl)-4-ylmethoxy)-3-hydroxy-2-(1-piperidinyl)cyclopentyl)-4-++++heptonoic acid) or YM 158 ((3-((4-tert-butylthiazol-2-yl)methoxy)-5′-(3-(4-chlorobenzenesulfonyl)propyl-2′-(1H-tetrazol-5-ylmethoxy)benzanilide monosodium salt monohydrate), and at least one nitric oxide donor (preferably L-arginine, N-hydroxy-L-arginine or S-nitroso-glutathione) with at least one (x-adrenergic receptor antagonist (preferably phentolamine, prazosin, doxazosin, terazosin, yohimbine and/or moxisylyte or a pharmaceutically acceptable salt thereof) and/or at least one phosphodiesterase inhibitors (preferably papaverine, zaprinast, sildenafil and/or IC 351 or a pharmaceutically acceptable salt thereof). Most preferred compositions comprising SQ 29548, a NO donor, and an α-adrenergic receptor antagonist, (preferably phentolamine or yohimbine) or a phosphodiesterase inhibitor (preferably sildenafil and/or ICI 351). The NO donor is preferably L-arginine or L-arginine glutamate. Phentolamine is preferably phentolamine hydrochloride or phentolamine mesylate, more preferably phentolamine mesylate. Yohimbine is preferably yohimbine hydrochloride, yohimbine tartarate, yohimbe bark powder or yohimbe bark extract, more preferably yohimbine hydrochloride or yohimbine tartarate. Sildenafil is preferably sildenafil hydrochloride or sildenafil citrate, more preferably sildenafil citrate.

Another embodiment of the present invention provides methods to prevent or treat ischemic heart disorders, myocardial infarction, angina pectoris, stroke, migraine, cerebral hemorrhage, cardiac fatalities, transient ischaemic attacks, complications following organ transplants, coronary artery bypasses, angioplasty, endarterectomy, atherosclerosis, pulmonary embolism, bronchial asthma, bronchitis, pneumonia, thrombotic or thromboembolic disorders, intrauterine growths, preganacy-induced hypertension, preeclamsia, eclamsia, circulatory shock of various organs, such as for example, hemorrhage, septicemia, heart failure, trauma, acute pancreatitis, burn and bacterial origin, nephritis, graft rejection and cancerous metastases; gastrointestinal disorders, or renal diseases and disorders; to reduce serum uric acid levels; to inhibit platelet aggregation or platelet adhesion; and to relax smooth muscles by administering to a patient in need thereof a therapeutically effective amount of at least one thromboxane inhibitor and at least one compound that donates, transfers or releases nitric oxide, elevates endogenous levels of endothelium-derived relaxing factor, or EDRF in vivo, stimulates endogenous synthesis of nitric oxide or is a substrate for nitric oxide synthase. These methods may further comprise administering at least one therapeutic agent. For example, the patient may be administered a therapeutically effective amount of at least one thromboxane inhibitor and at least one compound that donates, transfers or releases nitric oxide, elevates levels of endogenous EDRF or nitric oxide or is a substrate for nitric oxide synthase. In yet another embodiment, the patient may be administered a therapeutically effective amount of at least one thromboxane inhibitor, at least one therapeutic agent, and at least one compound that donates, transfers or releases nitric oxide, elevates endogenous levels of endothelium-derived relaxing factor, stimulates endogenous synthesis of nitric oxide or is a substrate for nitric oxide synthase. The “therapeutic agents” include, for example, vasoactive agents, nonsterodial antiinflammatory compounds, selective cyclooxygenase inhibitors, anticoagulants, angiotensin converting enzymes (ACE) inhibitors, angiotensin II receptor antagonists, renin inhibitors, and the like. Suitable vasoactive agents include those described herein. The thromboxane inhibitor, nitric oxide donors and/or therapeutic agents may be administered separately or as a composition. The compounds and compositions of the present invention can also be administered in combination with other medications used for the treatment of these diseases or disorders.

Suitable NSAIDs include, but are not limited to, acetaminophen, aspirin, diclofenac, ibuprofen, ketoprofen, naproxen and the like. Suitable NSAIDs are described more fully in the literature, such as in Goodman and Gilman, The Pharmacological Basis of Therapeutics (9th Edition), McGraw-Hill, 1995, Pgs. 617-657; the Merck Index on CD-ROM, Twelfth Edition, Version 12:1, 1996; STN express file reg and file phar. Suitable NSAIDs are also described and claimed in U.S. Pat. No. 6,057,347 and co-pending Application Ser. No. 09/429,019, filed Oct. 30, 1998, both of which are assigned to NitroMed, Inc., the disclosures of each of which are incorporated by reference herein in their entirety.

Suitable COX-2inhibitors include, but are not limited to, those disclosed in, for example, U.S. Pat. Nos. 5,681,842, 5,750,558, 5,756,531, 5,776,984 and in WO 97/41100, WO 98/39330, WO 99/10331, WO 99/10332 and WO 00/24719 assigned to Abbott Laboratories; and in WO 98/50075, WO 00/29022 and WO 00/29023 assigned to Algos Pharmaceutical Corporation; and in WO 99/15205 assigned to Almirall Prodesfarma S. A.; and in U.S. Pat. No. 5,980,905 assigned to AMBI Inc.; and in U.S. Pat. No. 5,945,538 assigned to American Cyanamid Company; and in U.S. Pat. Nos. 5,776,967, 5,824,699, 5,830,911 and in WO 98/04527 and WO 98/21195 assigned to American Home Products Corporation; and in WO 98/22442 assigned to Angelini Richerche S. P. A. Societa Consortile; and in U.S. Pat. No. 6,046,191 and in WO 99/18960 and WO 00/00200 assigned to Astra Pharmaceuticals Ltd.; and in U.S. Pat. No. 5,905,089 assigned to Board of Supervisors of Louisiana State University; and in WO 97/13767 assigned to Chemisch Pharmazeutische Forschungsgesellschaft MBH; and in WO 98/57924 and WO 99/61436 assigned to Chugai Seiyaku Kabushiki Kaisha; and in WO 00/13685 assigned to Cornell Research Foundation Inc.; and in WO 96/10021 assigned to The Du Pont Merck Pharmaceutical Company; and in EP 0 087 629 B 1 assigned to E. I. Du Pont de Nemours and Company; and in WO 99/13799 assigned to Euro-Celtique; and in U.S. Pat. No. 5,134,142 and in WO 91/19708, WO 97/13755, WO 99/15505, WO 99/25695 and in EP 0 418 845 B1 and EP 0 554 829 A2 assigned to Fujisawa Pharmaceutical Co. Ltd.; and in U.S. Pat. Nos. 5,344,991, 5,393,790, 5,434,178, 5,466,823, 5,486,534, 5,504,215, 5,508,426, 5,510,496, 5,516,907, 5,521,207, 5,563,165, 5,580,985, 5,596,008, 5,616,601, 5,620,999, 5,633,272, 5,643,933, 5,668,161, 5,686,470, 5,696,143, 5,700,816, 5,719,163, 5,753,688, 5,756,530, 5,760,068, 5,859,257, 5,908,852, 5,935,990, 5,972,986, 5,985,902, 5,990,148, 6,025,353, 6,028,072, 6,136,839 and in WO 94/15932, WO 94/27980, WO 95/11883, WO 95/15315, WO 95/15316, WO 95/15317, WO 95/15318, WO 95/21817, WO 95/30652, WO 95/30656, WO 96/03392, WO 96/03385, WO 96/03387, WO 96/03388, WO 96/09293, WO 96/09304, WO 96/16934, WO 96/25405, WO 96/24584, WO 96/24585, WO 96/36617, WO 96/38418, WO 96/38442, WO 96/41626, WO 96/41645, WO 97/11704, WO 97/27181, WO 97/29776, WO 97/38986, WO 98/06708, WO 98/43649, WO 98/47509, WO 98/47890, WO 98/52937, WO 99/22720, WO 00/23433, WO 00/37107, WO 00/38730, WO 00/38786 and WO 00/53149 assigned to G. D. Searle & Co.; and in WO 96/31509, WO 99/12930, WO 00/26216 and WO 00/52008 assigned to Glaxo Group Limited; and in EP 1 006 114 A1 and in WO 98/46594 assigned to Grelan Pharmaceutical Co. Ltd.; and in WO 97/34882 assigned to Grupo Farmaceutico Almirall; and in WO 97/03953 assigned to Hafslund Nycomed Pharma A G; and in WO 98/32732 assigned to Hoffman-La Roche A G; and in U.S. Pat. Nos. 5,945,539, 5,994,381, 6,002,014 and in WO 96/19462, WO 96/19463 and in EP 0 745 596 A1 assigned to Japan Tobacco, Inc.; and in U.S. Pat. Nos. 5,686,460, 5,807,873 and in WO 97/37984, WO 98/05639, WO 98/11080 and WO 99/21585 assigned to Laboratories USPA; and in WO 99/62884 assigned to Laboratories Del Dr. Esteve, S. A.; and in WO 00/08024 assigned to Laboratorios S.A.L.V.A.T., S. A.; and in U.S. Pat. Nos. 5,585,504, 5,840,924, 5,883,267, 5,925,631, 6,001,843, 6,080,876 and in WO 97/44027, WO 97/44028, WO 97/45420, WO 98/00416, WO 98/47871, WO 99/15503, WO 99/15513, WO 99/20110, WO 99/45913, WO 99/55830, WO 00/25779 and WO 00/27382 assigned to Merck & Co. Inc.; and in U.S. Pat. Nos. 5,409,944, 5,436,265, 5,474,995, 5,536,752, 5,550,142, 5,510,368, 5,521,213, 5,552,422, 5,604,253, 5,604,260, 5,639,780, 5,677,318, 5,691,374, 5,698,584, 5,710,140, 5,733,909, 5,789,413, 5,817,700, 5,840,746, 5,849,943, 5,861,419, 5,981,576, 5,994,379, 6,020,343, 6,071,936, 6,071,954 and in EP 0 788 476 B 1, EP 0 863 134 A1, EP 0 882 016 B1 and in WO 94/20480, WO 94/13635, WO 94/26731, WO 95/00501, WO 95/18799, WO 96/06840, WO 96/13483, WO 96/19469, WO 96/21667, WO 96/23786, WO 96/36623, WO 96/37467, WO 96/37468, WO 96/37469, WO 97/14691, WO 97/16435, WO 97/28120, WO 97/28121, WO 97/36863, WO 98/03484, WO 98/41511, WO 98/41516, WO 98/43966, WO 99/14194, WO 99/14195, WO 99/23087, WO 99/41224 and WO 00/68215 assigned to Merck Frosst Canada & Co., and in WO 99/59635 assigned to Merck Sharp & Dohme Limited; and in U.S. Pat. No. 5,380,738 assigned to Monsanto Company; and in WO 00/01380 assigned to A. Nattermann & Co.; and in WO 99/61016 assigned to Nippon Shinyaku Co. Ltd.; and in WO 99/33796 assigned to Nissin Food Products Co. Ltd.; and in WO 99/11605 assigned to Novartis A G; and in WO 98/33769 assigned to Nycomed Austria GMBIH; and in U.S. Pat. Nos. 6,077,869 and 6,083,969 and in WO 00/51685 assigned to Ortho-McNeil Pharmaceutical, Inc.; and in U.S. Pat. No. 5,783,597 assigned to Ortho Pharmaceutical Corporation; and in WO 98/07714 assigned to Oxis International Inc.; and in WO 00/10993 assigned to Pacific Corporation; and in EP 0 937 722 A1 and in WO 98/50033, WO 99/05104, WO 99/35130 and WO 99/64415 assigned to Pfizer Inc.; and in WO 00/48583 assigned to Pozen Inc.; and in U.S. Pat. No. 5,908,858 assigned to Sankyo Company Limited; and in WO 97/25045 assigned to Smithtkline Beecham Corporation; and in U.S. Pat. No. 5,399,357 assigned to Takeda Chemical Industries, Ltd.; and in WO 99/20589 assigned to The University of Sydney; and in U.S. Pat. No. 5,475,021 and WO 00/40087 assigned to Vanderbilt University; and in WO 99/59634 assigned to Wakamoto Pharmaceutical Co. Ltd., and in co-pending application Ser. No. 09/741,816 filed Dec. 23, 1999, and Application No. 60/256,932, filed Dec. 21, 2000, which are assigned to NitroMed, Inc., the disclosures of each of which are incorporated by reference herein in their entirety.

Suitable anticoagulants include, but are not limited to, heparin, coumarin, aspirin, protamine, warfarin, dicumarol, phenprocoumon, indan-1,3-dione, acenocoumarol, ansindione, and the like. Suitable anticoagulants are described more fully in the literature, such as in Goodman and Gilman, The Pharmacological Basis of Therapeutics (9th Edition), McGraw-Hill, 1995, Pgs. 1341-1359; the Merck Index on CD-ROM, Twelfth Edition, Version 12:1, 1996; STN express file reg and file phar.

Suitable angiotensin-converting enzyme inhibitors, include, but are not limited to, alacepril, benazepril, captopril, ceronapril, cilazapril, delapril, enalapril, enalaprilat, fosinopril, imidapril, lisinopril, moveltipril, perindopril, quinapril, ramipril, spirapril, temocapril, trandolapril, and the like. Suitable angiotensin-converting enzyme inhibitors are described more fully in the literature, such as in Goodman and Gilman, The Pharmacological Basis of Therapeutics (9th Edition), McGraw-Hill, 1995, Pgs. 733-838; and the Merck Index on CD-ROM, Twelfth Edition, Version 12:1, 1996; and on STN Express, file phar.

Suitable angiotensin II receptor antagonists, include, but are not limited to, ciclosidornine, eprosartan, furosemide, irbesartan, losartan, saralasin, valsartan, and the like. Suitable angiotensin II receptor antagonists are described more fully in the literature, such as in Goodman and Gilman, The Pharmacological Basis of Therapeutics (9th Edition), McGraw-Hill, 1995, Pgs. 733-838; and the Merck Index on CD-ROM, Twelfth Edition, Version 12:1, 1996; and on STN Express, file phar.

Suitable renin inhibitors, include, but are not limited to, enalkrein, RO 42-5892, A 65317, CP 80794, ES 1005, ES 8891, SQ 34017, and the like. Suitable renin inhibitors are described more fully in the literature, such as in Goodman and Gilman, The Pharmacological Basis of Therapeutics (9th Edition), McGraw-Hill, 1995, Pgs. 733-838; and the Merck Index on CD-ROM, Twelfth Edition, Version 12:1, 1996; and on STN Express, file phar.

When administered in vivo, the compositions of the present invention may be administered with pharmaceutically acceptable carriers and in dosages described herein. When the compositions of the present invention are administered as a mixture of at least one thromboxane inhibitor and at least one nitric oxide donor, they can also be used in combination with one or more additional compounds (e.g., therapeutic agents). When administered separately, the nitric oxide donor(s) and/or therapeutic agent may be administered simultaneously with, subsequently to, or prior to administration of the thromboxane inhibitor(s) and/or other additional compound(s) to prevent or treat the diseases described herein.

The compounds and compositions of the present invention can be administered by any available and effective delivery system including, but not limited to, orally, bucally, parenterally, by inhalation spray (oral or nasal), by topical application, by injection into the corpus cavernosum tissue, by transurethral drug delivery, vaginally, or rectally (e.g., by the use of suppositories) in dosage unit formulations containing conventional nontoxic pharmaceutically acceptable carriers, adjuvants, and vehicles, as desired. Parenteral includes subcutaneous injections, intravenous injections, intramuscular injections, intrasternal injections, and infusion techniques. Parenteral also includes injection into the corpus cavernosum tissue, which can be conducted using any effective injection system including, but not limited to, conventional syringe-and-needle systems or needleless injection devices.

Solid dosage forms for oral administration can include capsules, tablets, effervescent tablets, chewable tablets, pills, powders, effervescent powders, sachets, granules and gels. In such solid dosage forms, the active compounds can be admixed with at least one inert diluent such as sucrose, lactose or starch. Such dosage forms can also comprise, as in normal practice, additional substances other than inert diluents, e.g., lubricating agents such as magnesium stearate. In the case of capsules, tablets, effervescent tablets, and pills, the dosage forms can also comprise buffering agents. Soft gelatin capsules can be prepared to contain a mixture of the active compounds or compositions of the present invention and vegetable oil. Hard gelatin capsules can contain granules of the active compound in combination with a solid, pulverulent carrier such as lactose, saccharose, sorbitol, mannitol, potato starch, corn starch, amylopectin, cellulose derivatives of gelatin. Tablets and pills can be prepared with enteric coatings.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting agents, emulsifying and suspending agents, and sweetening, flavoring, and perfuming agents.

Suppositories for vaginal or rectal administration of the compounds and compositions of the invention can be prepared by mixing the compounds or compositions with a suitable nonirritating excipient such as cocoa butter and polyethylene glycols which are solid at room temperature but liquid at body temperature, such that they will melt and release the drug.

Injectable preparations, for example, sterile injectable aqueous or oleaginous suspensions can be formulated according to the known art using suitable dispersing agents, wetting agents and/or suspending agents. The sterile injectable preparation can also be a sterile injectable solution or suspension in a nontoxic parenterally acceptable diluent or solvent, for example, as a solution in 1,3-butanediol. Among the acceptable vehicles and solvents that can be used are water, Ringer's solution, and isotonic sodium chloride solution. Sterile fixed oils are also conventionally used as a solvent or suspending medium.

Topical administration, which is well known to one skilled in the art, involves the delivery of pharmaceutical agents via percutaneous passage of the drug into the systemic circulation of the patient. Topical administration includes vaginal administration, vulval administration, penile administration and rectal administration. Topical administration can also involve transdermal patches or iontophoresis devices. Other components can be incorporated into the transdermal patches as well. For example, compositions and/or transdermal patches can be formulated with one or more preservatives or bacteriostatic agents including, but not limited to, methyl hydroxybenzoate, propyl hydroxybenzoate, chlorocresol, benzalkoonium chloride, and the like.

Dosage forms for topical administration of the compounds and compositions of the present invention preferably include creams, sprays, lotions, gels, ointments, emulsions, coatings for condoms, liposomes, foams, and the like. Administration of the cream, spray, ointment, lotion, gel, emulsion, coating, liposome, or foam can be accompanied by the use of an applicator or by transurethral drug delivery using a syringe with or without a needle or penile insert or device, or by clitoral, vulval or vaginal delivery, and is within the skill of the art. Alternatively, the compositions may be contained within a vaginal ring, tampon, suppository, sponge, pillow, puff, or osmotic pump system; these platforms are useful solely for vaginal delivery. Typically a lubricant and/or a local anesthetic for desensitization can also be included in the formulation or provided for use as needed. Lubricants include, for example, K-Y jelly (available from Johnson & Johnson) or a lidocaine jelly, such as XYLOCAINE® 2% jelly (available from Astra Pharmaceutical Products). Local anesthetics include, for example, novocaine, procaine, tetracaine, benzocaine and the like.

Ointments are semisolid preparations that are typically based on petrolatum or other petroleum derivatives. The specific ointment base to be used, as will be appreciated by those skilled in the art, is one that will provide for optimum drug delivery An ointment base should be inert, stable, nonirritating and nonsensitizing. Ointment bases may be grouped in four classes: oleaginous bases; emulsifiable bases; emulsion bases; and water-soluble bases. Oleaginous ointment bases include, for example, vegetable oils, fats obtained from animals, semisolid hydrocarbons obtained from petroleum, and the like. Emulsifiable ointment bases, also known as absorbent ointment bases, contain little or no water and include, for example, hydroxystearin sulfate, anhydrous lanolin, hydrophilic petrolatum, and the like. Emulsion ointment bases are either water-in-oil (W/O) emulsions or oil-in-water (O/W) emulsions, and include, for example, cetyl alcohol, glyceryl monostearate, lanolin and stearic acid, and the like. In a particular embodiment, water-soluble ointment bases are preferred and are prepared from polyethylene glycols of varying molecular weight, and can be determined by standard techniques as described in Remington: The Science and Practice of Pharmacy.

Lotions are preparations that may be applied without friction, and are typically liquid or semiliquid preparations in which solid particles, including the active agent, are present in a water or alcohol base. Lotions are usually suspensions of solids, and in a particular embodiment, may comprise a liquid oily emulsion of the oil-in-water type. It is generally necessary that the insoluble matter in a lotion be finely divided. Lotions will typically contain suspending agents to produce better dispersions as well as compounds useful for localizing the active agent in contact with the skin, such as, for example, methylcellulose, sodium carboxymethyl-cellulose, and the like.

Emulsion formulations are generally formed from a dispersed phase (for example, a pharmacologically active agent), a dispersion medium and an emulsifing agent. If desired, emulsion stabilizers can be included in the formulation as well. A number of pharmaceutically useful emulsions are known in the art, including, for example, oil-in-water (o/w) formulations, water-in-oil (w/o) formulations and multiple emulsions such as w/o/w or o/w/o formulations. Emulsifying agents suitable for use in such formulations include, but are not limited to, TWEEN 60®, SPAN 80®, cetostearyl alcohol, myristyl alcohol, glyceryl monostearate, sodium lauryl sulfate, and the like.

Creams are, as known in the art, viscous liquid or semisolid emulsions, either oil-in-water or water-in-oil. Cream bases are water-washable, and contain an oil phase, an emulsifier and an aqueous phase. The oil phase, also sometimes called the “internal” phase, is generally comprised of petrolatum and a fatty alcohol such as, cetyl alcohol, stearyl alcohol, and the like; the aqueous phase usually, although not necessarily, exceeds the oil phase in volume, and generally contains a humectant. The emulsifier in a cream formulation is generally a nonionic, anionic, cationic or amphoteric surfactant.

The ointments, lotions, emulsions and creams are formed by dispersing finely divided or dissolved the thromboxane inhibitor(s), nitric oxide donor(s) and/or vasoactive agent(s) uniformly throughout the vehicle or base using conventional techniques, typically by levigating the compound with a small quantity of the base to form a concentrate which is then diluted geometrically with further base. Alternatively, a mechanical mixer may be used. Creams, lotions and emulsions are formed by way of a two-phase heat system, wherein oil-phase ingredients are combined under heat to provide a liquified, uniform system. The aqueous-phase ingredients are separately combined using heat. The oil and aqueous phases are then added together with constant agitation and allowed to cool. At this point, concentrated agents may be added as a slurry. Volatile or aromatic materials can be added after the emulsion has sufficiently cooled. Preparation of such pharmaceutical compositions is within the general skill of the art. See, e.g., Remington's Pharmaceutical Sciences, 18th Ed. (Easton, Pa.: Mack Publishing Company, 1990).

The compounds of the present invention can also be incorporated into gel formulations using known techniques. Two-phase gel systems generally comprise a suspension or network of small, discrete particles interpenetrated by a liquid to provide a dispersed phase and a liquid phase. Single-phase gel systems are formed by distributing organic macromolecules uniformly throughout a liquid such that there are no apparent boundaries between the dispersed and liquid phases. Suitable gelling agents for use herein include synthetic macromolecules, such as, CARBOMERS®, polyvinyl alcohols, and polyoxyethylene-polyoxypropylene copolymers, and the like; gums such as, tragacanth, as well as sodium alginate, gelatin, methylcellulose, sodium carboxymethylcellulose, methylhydroxyethyl cellulose, hydroxyethyl cellulose, and the like. In order to prepare a uniform gel, dispersing agents such as alcohol or glycerin may be added, or the gelling agent may be dispersed by trituration, mechanical mixing or stiffing, or combinations thereof.

The compounds and compositions of the present invention will typically be administered in a pharmaceutical composition containing one or more carriers or excipients, i.e., pharmaceutically acceptable organic or inorganic carrier substances suitable for parenteral application which do not deleteriously react with the active compounds. Examples of pharmaceutically acceptable carriers include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicie acid, viscous paraffin, perfume oil, fatty acid monoglycerides and diglycerides, petroethral fatty acid esters, hydroxymethyl-cellulose, polyvinylpyrrolidone, and the like.

The pharmaceutical preparations can be sterilized and if desired, mixed with auxiliary agents which do not deleteriously react with the active compounds, e.g., lubricants, preservatives, stabilizers, wetting agents, emulsifiers, salts for influencing osmotic pressure, buffers, colorings, flavoring and/or aromatic substances, and the like. For parenteral application, particularly suitable vehicles consist of solutions, preferably oily or aqueous solutions, as well as suspensions, emulsions, or implants. Aqueous suspensions may contain substances which increase the viscosity of the suspension and include, for example, sodium carboxymethyl cellulose, sorbitol and/or dextran. Optionally, the suspension may also contain stabilizers.

The pharmaceutical compositions may also include a detergent in the formulation, in an amount effective to increase solubility of the thromboxane inhibitor, nitric oxide donor and/or vasoactive agent in the vehicle and bioavailability of the agent following administration. The detergent will typically be a nonionic, anionic, cationic or amphoteric surfactant. The surfactant is selected such that local irritation at the site of administration is avoided. Surfactants include, for example, TERGITOL® and TRITON® surfactants (Union Carbide Chemicals and Plastics, Danbury, Conn. polyoxyethylene sorbitan fatty acid esters, e.g., TWEEN® surfactants (Atlas Chemical Industries, Wilmington, Del.), such as, for example, polyoxyethylene 20 sorbitan monolaurate (TWEEN® 20), polyoxyethylene (4) sorbitan monolaurate (TWEEN® 21), polyoxyethylene 20 sorbitan monopalmitate (TWEEN® 40), polyoxyethylene 20 sorbitan monooleate (TWEEN® 80, and the like; polyoxyethylene 4 lauryl ether (BRIJ® 30), polyoxyethylene 23 lauryl ether (BRIJ 35), polyoxyethylene 10 oleyl ether (BRIJ® 97); polyoxyethylene glycol esters, such as, for example, poloxyethylene 8 stearate (MYRJ® 45), poloxyetllylene 40 stearate (MYRJ® 52) polyoxyethylene alkyl ethers, and the like; or mixtures thereof

The pharmaceutical preparation may also include one or more permeation enhancers. Permeation enhancers include those generally useful in conjunction with topical, transdermal or transmucosal drug delivery. Permeation enhancers include, for example, dimethylsulfoxide (DMSO), dimethyl formamide (DMF), N,N-dimethylacetamide (DMA), decylmethylsulfoxide (C₁₀ MSO), polyethylene glycol monolaurate (PEGML), polyethyleneglycol, glycerol monolaurate, lecithin, the 1-substituted azacycloheptan-2-ones, such as, 1-n-dodecylcyclazacycloheptan-2-one (available under the trademark AZONE® from Nelson Research & Development Co., Irvine, Calif.), lower alkanols (e.g., ethanol), C₆ to C₂₀-hydrocarbyl substituted 1,3-dioxane, C₆ to C₂₀-hydrocarbyl substituted 1,3-dioxolane and C₆ to C₂₀-hydrocarbyl substituted acetal, such as, SEPA® (available from Macrochem Co., Lexington, Mass.), alkonates, such as, alkyl-2-(N,N-disubstituted amino)-alkonate ester, N,N-disubstituted amino)-alkanol alkanoate, and the like, glycerides, such as mono, di and triglycerides and mixtures thereof, such as for example MIGLYOL® (Condea Vista Company, Houston, Tex.) and the like; polyglycolyzed glycerides, such as, for example, LABRASOL® and LABRAPIL®, and the like; and surfactants as discussed above, including, for example, TERGITOL,® and TRITON® surfactants, NONOXYNOL-9® and TWEEN-80®. In particular embodiments the penetration enhancers may be MIGLYOL®, LABRASOL® or LABRAFIL®, including mixtures thereof.

In some cases, the formulations may include one or more compounds effective to inhibit enzymes present in the vaginal or vulvar areas which could degrade or metabolize the pharmacologically active agent. For example, with a prostaglandin as the vasoactive agent, it may be preferred to include an effective inhibiting amount of a compound effective to inhibit prostaglandin-degrading enzymes. Such compounds include, for example, fatty acids, fatty acid esters, and NAD inhibitors.

Various delivery systems are known and can be used to administer the compounds or compositions of the present invention, including, for example, encapsulation in liposomes, microbubbles, emulsions, microparticles, microcapsules and the like. The required dosage can be administered as a single unit or in a sustained release form.

The bioavailabilty of the compositions can be enhanced by micronization of the formulations using conventional techniques such as grinding, milling, spray drying and the like in the presence of suitable excipients or agents such as phospholipids or surfactants. The bioavailability and absorption of the thromboxane inhibitor, nitric oxide donor and/or vasoactive agent can be increased by the addition of tabletting excipients, such as, for example β-cyclodextrin, a β-cyclodextrin derivative, such as for example, hydroxypropyl-β-cyclodextrin (HPBCD), and the like. Inclusion complexes are complexes formed by interaction of macrocyclic compounds containing an intramolecular cavity of molecular dimensions with the smaller, pharmacologically active agent. Preferred inclusion complexes are formed from α-, β- and γ-cyclodextrins, or from clathrates, in which the “host” molecules form a crystal lattice containing spaces in which “guest” molecules (i.e., in this case, the nitric oxide donor and/or vasoactive agent) will fit. See, e.g., Hagan, Clathrate Inclusion Compounds (New York: Reinhold, 1962).

Liposomes are microscopic vesicles having a lipid wall comprising a lipid bilayer, and can be used as drug delivery systems as well. Generally, liposome formulations are preferred for poorly soluble or insoluble pharmaceutical agents. Liposomal preparations for use in the pressent invention include cationic (positively charged), anionic (negatively charged) and neutral preparations. Cationic liposomes are readily available. For example, N(1-2,3-dioleyloxy)propyl)-N,N,N-triethylammonium (DOTMA) liposomes are available under the tradename LIPOFECTIN® (GIBCO BRL, Grand Island, N.Y.). Similarly, anionic and neutral liposomes are readily available as well, from Avanti Polar Lipids (Birmingham, Ala.), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), dioleoylphoshatidyl ethanolamine (DOPE), and the like. These materials can also be mixed with DOTMA in appropriate ratios. Methods for making liposomes using these materials are well known in the art. See Remington's Pharmaceutical Sciences, supra.

The release of the thromboxane inhibitor, nitric oxide donor and/or vasoactive agent can be controlled by dissolution (bioerosion) of a polymer using either encapsulated dissolution control or matrix dissolution control. In encapsulated dissolution control, the compound is coated with a membrane of slowly dissolving polymeric or wax materials. When the encapsulating membrane has dissolved, the agent core is available for immediate release and adsorption across the epithelial or mucosal surfaces of the vagina or vulvar area. Biocrodible coating materials may be selected from a variety of natural and synthetic polymers, depending on the agent to be coated and the desired release characteristics. Exemplary coating materials include gelatins, carnauba wax, shellacs, ethylcellulose, cellulose acetate phthalate, cellulose acetate butyrate, and the like. Release of the compound is controlled by adjusting the thickness and dissolution rate of the polymeric membrane. A uniform sustained release can be attained by compressing a population of particles of the agent with varying membrane thickness (e.g., varying erosion times) into a tablet form for a single administration.

In matrix dissolution control, the thromboxane inhibitor, nitric oxide donor and/or vasoactive agent is dissolved or dispersed within a matrix of, such as, for example, an erodible wax. The compound is released for adsorption across the epithelial or mucosal surfaces of the vagina or vulvar area as the matrix bioerodes. The rate of compound availability is generally controlled by the rate of penetration of the dissolution media (i.e., vaginal fluids) into the matrix, wherein the rate of penetration is dependent on the porosity of the matrix material. Bioerodible matrix dissolution delivery systems can be prepared by compressing the nitric oxide donor and/or vasoactive agent with a slowly soluble polymer carrier into a tablet or suppository form. There are several methods of preparing drug/wax particles including congealing and aqueous dispersion techniques. In congealing methods, the vasodilating agent is combined with a wax material and either spray-congealed, or congealed and then screened. For an aqueous dispersion, the vasodilating agent/wax combination is sprayed or placed in water and the resulting particles collected. Matrix dosage formulations can be formed by compaction or compression of a mixture of vasodilating agent, polymer and excipients.

In an alternative embodiment, the compositions of the present invention may be administered as biodegradable adhesive film or sheet which adhere to the vulvar area. Such drug delivery systems are generally composed of a biodegradable adhesive polymer based on a polyurethane, a poly(lactic acid), a poly(glycolic acid), a poly(ortho ester), a polyanhydride, a polyphosphazene, or a mixture or copolymer thereof. Preferred biodegradable adhesive polymers include, for example, polyurethanes and block copolyurethanes containing peptide linkages, simple mixtures of polyurethanes and polylactides, and copolymers of acrylates and mono- or disaccharide residues.

The compounds and compositions of the present invention can be formulated as pharmaceutically acceptable salts. Pharmaceutically acceptable salts include, for example, alkali metal salts and addition salts of free acids or free bases. The nature of the salt is not critical, provided that it is pharmaceutically acceptable. Suitable pharmaceutically acceptable acid addition salts may be prepared from an inorganic acid or from an organic acid. Examples of such inorganic acids include, but are not limited to, hydrochloric, hydrobromic, hydroiodic, nitric (nitrate salt), nitrous (nitrite salt), carbonic, sulfuric and phosphoric acid and the like. Appropriate organic acids include, but are not limited to, aliphatic, cycloaliphatic, aromatic, heterocyclic, carboxylic and sulfonic classes of organic acids, such as, for example, formic, acetic, propionic, succinic, glycolic, gluconic, lactic, malic, tartaric, citric, ascorbic, glucuronic, maleic, fulmaric, pymuvic, aspartic, glutamic, benzoic, anthranilic, mesylic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesuifonic, sulfanilic, stearic, algenic, β-hydroxybutyric, cyclohexylaminosulfonic, galactaric and galacturonic acid and the like. Suitable pharmaceutically acceptable base addition salts include, but are not limited to, metallic salts made from aluminum, calcium, lithium, magnesium, potassium, sodium and zinc or organic salts made from primary, secondary and tertiary amines, cyclic amines, N,N′-dibenzylethylenediamine, chloroprocaine, choline, diethanolamine, ethylenediamine, meglumine (N-methylglucamine) and procaine and the like. All of these salts may be prepared by conventional means from the corresponding compound by reacting, for example, the appropriate acid or base with the compound.

“Therapeutically effective amount” refers to the amount of the thromboxane inhibitor, nitric oxide donor and/or therapeutic agent which is effective to achieve its intended purpose. While individual patient needs may vary, determination of optimal ranges for effective amounts of each nitric oxide donor is within the skill of the art. Generally the dosage regimen for treating a condition with the compounds and/or compositions of this invention is selected in accordance with a variety of factors, including the type, age, weight, sex, diet and medical condition of the patient, the severity of the dysfunction, the route of administration, pharmacological considerations such as the activity, efficacy, pharmacokinetic and toxicology profiles of the particular compound used, whether a drug delivery system is used, and whether the compound is administered as part of a drug combination and may be adjusted by one skilled in the art. Thus, the dosage regimen actually employed may vary from the preferred dosage regimen set forth herein.

The amount of a given thromboxane inhibitor which will be effective in the treatment of a particular dysfunction or condition will depend on the nature of the dysfunction or condition, and may be determined by standard clinical techniques, including reference to Goodman and Gilman, supra; The Physician's Desk Reference, supra; Medical Economics Company, Inc., Oradell, N.J., 1995; and Drug Facts and Comparisons, Inc., St. Louis, Mo., 1993. The precise dose to be used in the formulation will also depend on the route of administration, and the seriousness of the dysfunction or disorder, and should be decided by the physician and the patient's circumstances. Effective doses may be extrapolated from dose-response curves derived from in vitro or animal model test systems and are in the same ranges or less than as described for the commercially available compounds in the Physician's Desk Reference, supra.

Particularly preferred methods of administration of the thromboxane inhibitors for the treatment of male sexual dysfunction are by oral administration, by topical application, by injection into the corpus cavernosum, by transurethral administration or by the use of suppositories. The preferred methods of administration for female sexual dysfunction are by oral administration, topical application or by the use of suppositories.

The dose of the thromboxane inhibitor, in single or divided doses, in the pharmaceutical composition may be in amounts of about 0.1 mg to about 2500 mg and the actual amount administered will be dependent on the specific thromboxane inhibitor and if it is used in combination with other compounds. For example, when a thromboxane inhibitor is administered in combination with a NSAID, the dose of the thromboxane inhibitor is from about 0.1 mg to about 2500 mg, preferably about 2 mg to about 2000 mg, and the dose of the NSAID is preferably about 5 mg to about 1500 mg.

The doses of nitric oxide donors in the pharmaceutical composition may be in amounts of about 0.001 mg to about 30 g and the actual amount administered will be dependent on the specific nitric oxide donor compound. For example, when L-arginine is the nitric oxide donor, L-arginine may be administered orally in an amount of about 0.25 grams to about 10 grams (equivalent to about 0.5 grams to about 20 grams of L-arginine glutamate), preferably about 2 grams to about 4 grams (equivalent to about 4 grams to about 8 grams of L-arginine glutamate); more preferably about 2.5 grams to about 3.5 grams (equivalent to about 5 grams to about 7 grams of L-arginine glutamate); most preferably about 3 grams (equivalent to 6 grams of L-arginine glutamate).

The α-antagonist, such as phenfolamine, may be administered in amounts of about 3.7 mg to about 90 mg (equivalent to about 5 mg to about 120 mg phentolamine mesylate), preferably about 22 mg to about 37 mg (equivalent to about 30 mg to about 50 mg phentolamine mesylate), more preferably about 26 mg to about 34 mg (equivalent to about 35 mg to about 45 mg phentolamine mesylate), even more preferably about 28 mg to about 31 mg (equivalent to about 38 mg to about 42 mg phentolamine mesylate), most preferably about 30 mg (equivalent to about 40 mg phentolamine mesylate).

The α-antagonist, such as yohimbine, may be administered in an amount of about 1.0 mg to about 18.0 mg (equivalent to about 1.1 mg to about 19.8 mg yohimbine hydrochloride), preferably about 4.5 mg to about 6.4 mg, (equivalent to about 5.0 mg to about 7.0 mg yohimbine hydrochloride), more preferably about 5.0 mg to about 6.0 mg, (equivalent to about 5.5 mg to about 6.5 mg yohimbine hydrochloride), most preferably about 5.5 mg (equivalent to about 6.0 mg yohimbine hydrochloride ). The yohimbine can also be administered as its pharmaceutical salt, yohimbine tartarate, or yohimbe bark powder or extract that has been standardized to deliver up to about 18 mg of yohimbine.

The present invention also provides pharmaceutical kits comprising one or more containers filled with one or more of the ingredients of the pharmaceutical compounds and/or compositions of the present invention, including, at least one thromboxane inhibitor, one or more NO donors, and one or more therapeutic agents described herein. Such kits can also include, for example, other compounds and/or compositions (e.g., permeation enhancers, lubricants, and the like), a device(s) for administering the compounds and/or compositions, and written instructions in a form prescribed by a governmental agency regulating the manufacture, use or sale of pharmaceuticals or biological products, which instructions can also reflects approval by the agency of manufacture, use or sale for human administration.

### EXAMPLES

The following non-limiting examples are for purposes of illustration only and are not intended to limit the scope of the invention or claims.

In the examples, PGE₁ (specifically alprostadil) was obtained from Upjohn (Belgium). The drugs were dissolved in distilled water, except PGE₁ and PGE₀, which were dissolved at a concentration of 10 mM in ethanol. Dilutions were made in distilled water at the time of the experiment.

### Example 1

Relaxation of Human Corpus Cavernosum Tissue

Human corpus cavernosum specimens were obtained from impotent men at the time of penile prosthesis insertion. Tissues were maintained at 4-6° C. in M-400 solution, pH 7.4 (composition per 100 ml: mannitol, 4.19 g; KH₂PO₄, 0.205 g; K₂HPO₄.3H₂O, 0.97 g; KCl, 0.112 g; NaHCO₃, 0.084 g) until used. Corpus cavernosum tissues were typically used between 2 and 16 hours from extraction.

Measurement of the relaxation of corpus cavemosal tissue was carried out as follows. Corpus cavernosal tissue strips (3×3×7 mm) were immersed in a 8 ml organ chamber containing physiological salt solution, maintained at 37° C. and aerated with 5% CO₂/95% air, pH 7.4. The tissues were then contracted with phenylephrine (1 μM) and the relaxation response was evaluated after the cumulative addition of the compound to the organ chamber.

In experiments where the effect of the thromboxane A₂ receptor antagonist, SQ 29548 , was measured, SQ 29584 was added 20 minutes before the addition of phenylephrine. In experiments where the effect of the thromboxane A₂ agonist, U 46619 was measured, U 46619 was added after the phenylephrine-induced contractions stabilized, and then prostaglandin PGE₁ was added 10 minutes later, if required.

The data are expressed as mean i standard error of the percentage of total relaxation induced by 100 μM papaverine hydrochloride. Statistical analysis were conducted using a two-factor ANOVA statistical analysis using StatView software for Apple computers.

### Example 2

Comparison of the Relaxation Response of PGE₁ and PGE₀ in Human

Corpus Cavernosum Tissues

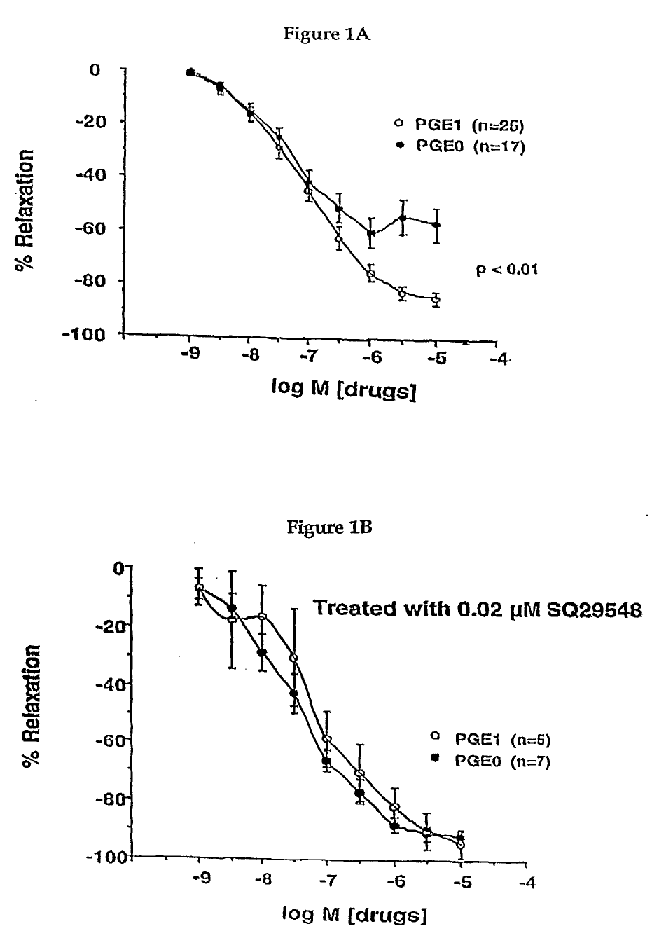

The tissues were prepared according to Example 1. The percent relaxation induced by either increasing concentrations of PGE₁ alone or increasing concentrations of PGE₀ alone in the absence (FIG. 1A) or presence of 0.02 μM SQ 29548 (FIG. 1B) were measured as described in Example 1. As can be seen from FIG. 1A, there was a significant contraction of the tissue in the presence of the higher concentrations of PGE₀ . However, after treatment with the thromboxane A₂receptor antagonist, SQ 29548 , (FIG. 1B) PGE₁ and PGE₀ were equipotent in relaxing human corpus carvenosum tissue.

### Example 3

Relaxation Response of PGE₀ in Human Corpus Cavernosum Tissues in the Presence of a Thromboxane A₂ Receptor Antagonist.

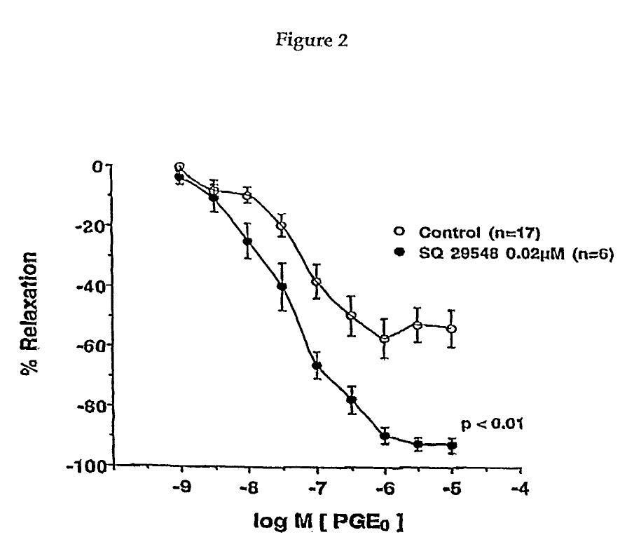

The tissues were prepared according to Example 1. The percent relaxation induced by either increasing concentrations of PGE₀ alone or in the presence of 0.02 M SQ 29548 were measured as described in Example 1. As can be seen from FIG. 2, there was a significant relaxation of the tissue in the presence of the thromboxane A₂ receptor antagonist, SQ 29548 (0.02 μM). These results indicate that the relaxation of the corpus cavernosum smooth muscle is mediated by the thromboxane receptor.

### Example 4

Relaxation Response of PGE₁ in Human Corpus Cavernosum Tissues in the Presence of a Thromboxane A₂ Receptor Agonist.

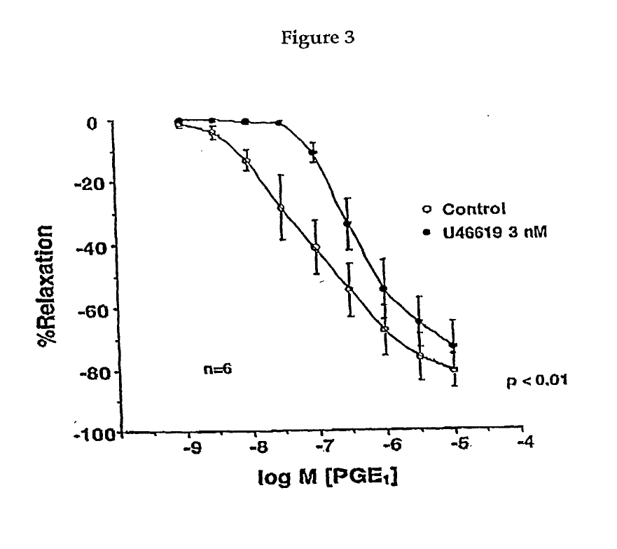

The tissues were prepared according to Example 1. The percent relaxation induced by either increasing concentrations of PGE₀ alone or in the presence of 0.003 μM U 46619 were measured as described in Example 1. As can be seen from FIG. 3, there was a significant contraction of the tissue in the presence of the thromboxane A₂ receptor agonist, U 46619 (0.003 μM). These results indicate that the relaxation of the corpus cavernosum smooth muscle is mediated by the thromboxane receptor.

### Example 5

Relaxation Response of PGE₁ or PGE₀ in Human Corpus Cavernosum Tissues from Diabetic or Non-diabetic Patients.

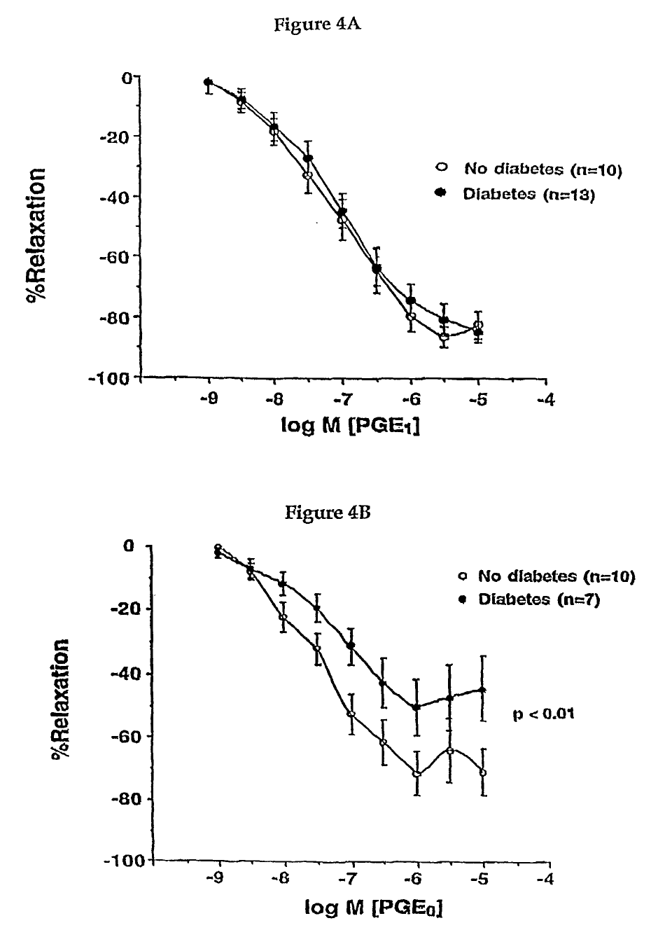

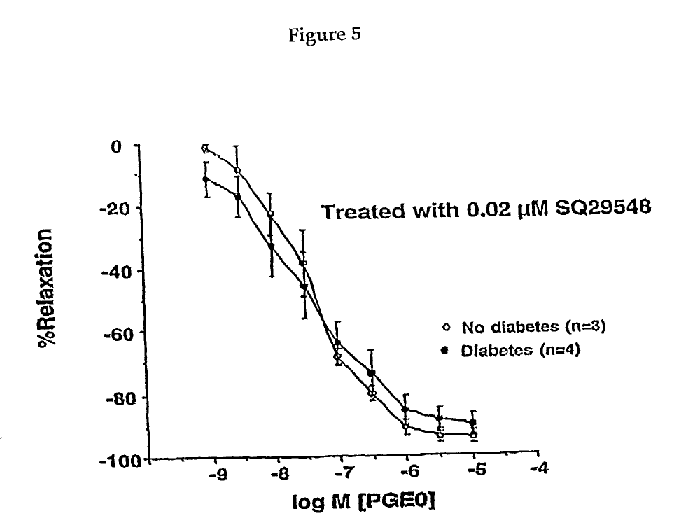

The tissues were prepared according to Example 1. The percent relaxation induced by either increasing concentrations of PGE₁ alone or PGE₀ alone in tissue obtained from diabetic or non-diabetic patients were measured as described in Example 1. As can be seen from FIG. 4A, at increasing concentrations of PGE₁, there was no difference in the relaxation response for tissues obtained from diabetic or non-diabetic patients. However, as can be seen from FIG. 4B, at increasing concentrations of PGE₀, there was a significant reduction in the relaxation of the tissue from diabetic patients compared to non-diabetic patients. This reduction in the relaxation of the tissue was eliminated by blocking the thromboxane A₂ receptor by the addition of 0.02 μM SQ 29548 (FIG. 5). These results indicate diabetic patients might have an overactive thromboxane-mediated effect.

### Example 6

Comparison of the Contratile Response of Human Corpus Cavernosum Tissues from Diabetic and Non-diabetic Patients

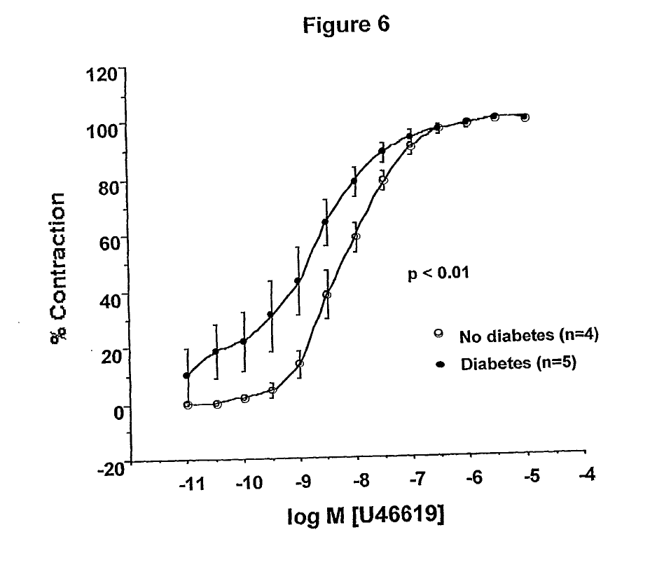

The tissues were prepared according to Example 1. The percent contraction response induced by increasing concentrations of the thromboxane agonist U 46619 were measured as described in Example 1. As can be seen from FIG. 6, there was a significant contraction of the tissue obtained from diabetic patients in the presence of U 46619 . These results indicate that the contraction of the corpus cavernosum smooth muscle in diabetic patients is mediated by the thromboxane receptor.

### Example 7

Relaxation Response of PGE₀ in Human Corpus Cavernosum Tissues from Hypertensive or Non-hypertensive Patients.

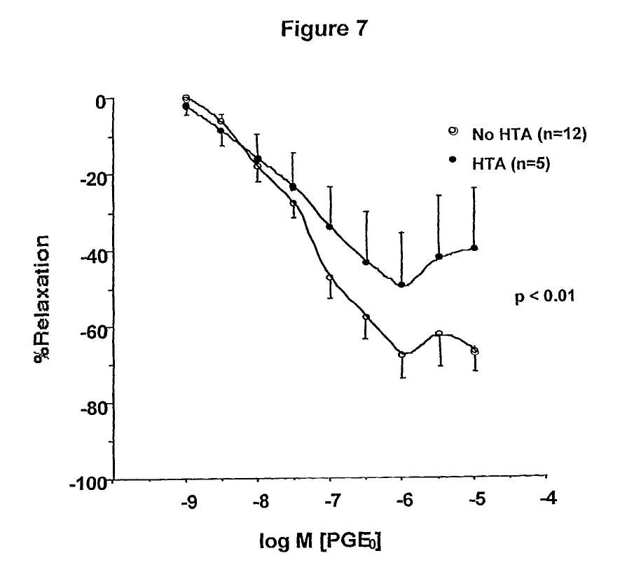

The tissues were prepared according to Example 1. The percent relaxation induced by either increasing concentrations of PGE₀ in tissue obtained from hypertensive or non-hypertensive patients were measured as described in Example 1. As can be seen from FIG. 7, at increasing concentrations of PGE₀, there was a significant reduction in the relaxation of the tissue from hypertensive patients compared to non-hypertensive patients. These results indicate hypertensive patients might have an overactive thromboxane-mediated effect.

### Example 8

Relaxation of Human Corpus Cavernosum Resistant Arteries

Human corpus cavernosum tissues were prepared according to Example 1. Penile small arteries, helicine arteries (lumen diameter 150-400 μ), which are the terminal branches of deep penile arteries, were dissected by carefully removing the adhering trabecular tissue. Arterial ring segments (2 mm long) were subsequently mounted on two 40 μm wires on microvascular double Halpern-Mulvany myographs (J. P. Trading, Aarhus, Denmark) for isometric tension recordings.

Measurement of the relaxation of corpus cavernosal resistant arteries was carried out as follows. The segments were allowed to equilibrate for 30 min in physiological salt solution of the following composition (mM): NaCl 119, KCl 4.6, CaCl₂ 1.5, MgCl₂ 1.2, NaHCO₃ 24.9, glucose 11, KH₂PO₄ 1.2, EDTA 0.027 at 37° C. continuously bubbled with 95% O₂/5% CO₂ mixture to maintain a pH of 7.4. Passive tension and internal circumference of vascular segments when relaxed in situ under a transmural pressure of 100 mmHg (L₁₀₀). The arteries were then set to an internal circumference equivalent to 90% of L₁₀₀, such that the force development was close to maximal (Mulvany and Halpern, Circ. Res., 41:19-26 (1977)).

The segments were then exposed to 125 mM K⁺ (physiological salt solution potassium salt form, by the equimolar substitution of NaCl for KCl in physiological salt solution) and the contraction response was measured. The arteries were contracted with 1 μM norepinephrine and relaxation responses were evaluated by cumulative additions of the acetylcholine (1 nM to 3 μ) to the organ chamber. The segments were washed with fress physiological salt solution, treated with the thromboxane inhibitor SQ 29548 (0.02 μM) and the response to acetylcholine measured again.

### Example 9

Relaxation Response of Acetylcholine in Human Corpus Resistant Arteries in the Presence of a Thromboxane A₂ Receptor Antagonist.

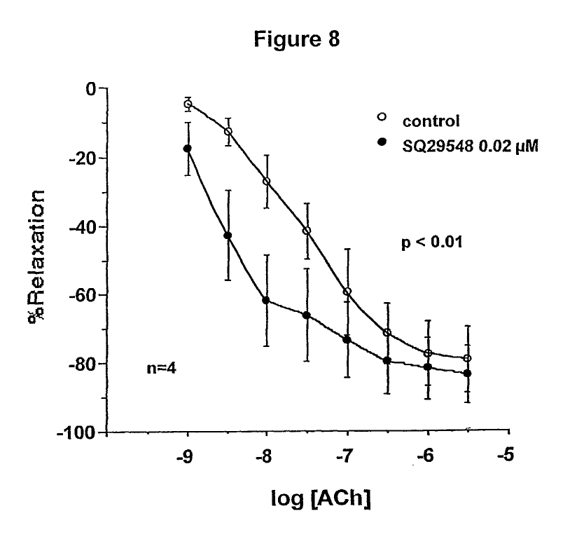

The segments were prepared according to Example 8. The percent relaxation induced by either increasing concentrations of acetylcholine alone or in the presence of 0.02 μM SQ 29548 were measured as described in Example 8. As can be seen from FIG. 8, there was a significant relaxation of the tissue in the presence of the thromboxane A₂ receptor antagonist, SQ 29548 (0.02 μM). These results indicate that the relaxation of the corpus cavernosum arteries is mediated by the thromboxane receptor.

The disclosure of each patent, patent application and publication cited or described in the specification is hereby incorporated by reference herein in its entirety.

Although the invention has been set forth in detail, one skilled in the art will appreciate that numerous changes and modifications can be made to the invention without departing from the spirit and scope thereof.

What is claimed is:

1. A method for preventing or treating a sexual dysfunction in a patient in need thereof comprising administering to the patient a therapeutically effective amount of at least one thromboxane inhibitor or a pharmaceutically acceptable salt thereof.

2. The method of claim 1, wherein the at least one thromboxane inhibitor is 2-(acetyloxy)-benzoic acid, 4-hydroxy-(Z)-(2,4,5-trimethyl-3,6-dioxo-1,4-cyclohexa dien-1-yl)-benezeneheptanoic acid, (1α(Z),2β,5α)-(±)-7-(5-(((1,1′-biphenyl)-4-yl)methoxy)-2-(4-morpholinyl)-3-oxocyclopentyl)-4-heptenoic acid, 3R-((4-fluorophenyl)sulfonyl)amino)-1,2,3,4-tetrahydro-9H-carbazole-9-propanoic acid, (4-(2-((phenylsulfonyl)amino)ethyl)phenoxy)-acetic acid, 4-(2-(((4-chlorophenyl)sulfonyl)amino)ethyl)-benzeneacetic acid, ((+)-1S-(1α,2α,3α,4α)-2-((3-(4-(n-pentylamino)carbonyl)-2-oxazolyl)-7-oxabicyclo(2.2.1)hept-2-yl)methyl)benzeneproponic acid, (8R,8′S)-4,4′-dihydroxy-3,3′-dimethoxy-7-oxo-8,8′-neolignan, 3-methyl-2-(3-pyridyl)indole-1-octanoic acid, CGS 15435, 4-(((4-chlorophenyl)sulfonyl)amino)butyl)-3-pyridine heptanoic acid, ((E)-7-phenyl-7-(3-pyridinyl)-6-heptenoic acid), dazmegrel, 4-(2-(1H-imidazol-1-yl)ethoxy)benzoic acid, (E)-6-(4-2-(4-chlorobenzene sulphonyl amino)-ethyl)phenyl)-6-(3-pyridyl)-hex-5-enoic acid, 2-(2-(4-chlorophenoxy)-2-methyl-1-oxopropoxy)-3-pyridinecarboxylic acid ethyl ester, (1α,2β(Z),3α,4α)-7-(3-((((phenylamino)carbonyl)hydrazono)methyl)bicyclo(2.2.1hept-2-yl), 7-((1S,2S,3S,4R)-3-(1-(3-(phenylthioureidoimino)ethyl)-bicyclo(2.2.1)heptane-2-yl)-5-heptenoic acid, 1-(((5-(4-chlorophenyl)-2-furanyl)methylene)amino)-3-(4-(1-piperazinyl)butyl)-2,4-imidazolidinedione, (E)-(±)-5-(((1-cyclohexyl-2-(1H-imidazol-1-yl)-3-phenyl propylidene)amino)oxypentanoic acid, 2,3-dihydro-5-(2-(1H-imidazol-1-yl)ethoxy)-indene-1-carboxylic acid, (5Z)-6-((2S,4R)-4-((4-chlorophenyl)sulfonyl)amino)-1-(3-pyridinylmethyl)-2-pyrrolidinyl)-5-hexenoic acid monohydrate, furegrelate, (1R-(1α(Z),2β,3β,5α))-(+)-7-(5-((1,1′-biphenyl)-4-ylmethoxy)-3-hydroxy-2-(1-piperidinyl)cyclopenty)-4-++++heptonoic acid, (±)-(1α(Z),2β,5β)-6-((2-(hexahydro-1H-azepin-1-yl)-5-((4-(4-propyl-3-pyridinyl)phenyl)methoxy)cyclopentyl)oxy)-4-hexanoic acid, (5E)-6-(3-(2-((4-iodophenyl)sulfonyl)amino)ethyl)phenyl)-6-(3-pyridinyl)-5-hexenoic acid, (5E)-6-(3-(2-((4-iodophenyl)sulfonyl)amino)-1,1-dimethylethyl)phenyl)-6-(3-pyridinyl)-5-hexenoic acid, 2-(6-carboxyhexyl)-3-n hexylcyclohexylaniine, (E)-7-phenyl-7-(3-pyridinyl)-6-heptenoic acid, 5(Z)-7-(2,2-dimethyl-4-(2-hydroxyphenyl)-1,3-dioxan-cis-5-yl)heptenoic acid, (Z)-(2α,4α,5α)-6-(2-chlorophenyl)-4-(2-hydroxyphenyl)-1,3-dioxan-5-yl)-4-hexenoic acid, 3-(4-(((4-chlorophenyl)sulfonyl)amino)butyl)-6-(1-methylethyl)-1-azulenesulfonic acid monosodium salt, (E)-11-(2-(5,6-dimethyl-1H-benzimidazol-1-yl)-6,11-dihydro-dibenz(b,e)oxepin-2-carboxylic acid sodium salt, sodium (E)-11-(2-(5,6-dimethyl-1-benzimidazolyl)ethylidene)-6,11-dihydrodibenz (b,e)oxepin-2-carboxylate monohydrate, 4-methyl-benzenesulfonate-N-((phenyhnethoxy)carbonyl)-serine ethyl ester, dibenzo(b,f)thiepin-3-methanol-5,5-dioxide, (−)-6,8-difloro-2,3,4,9-tetrahydro-9-((4-(methylsulfonyl)phenyl)methyl)-1H-carbazole-1-acetic acid, (5,5-dioxide-dibenzo(b,f) thiepin-3-carboxylic acid, 3-methyl-1-((4-chlorophenyl)methyl)-5-fluoro-α,α,-1H-indole-2-propanoic acid, midazogrel, (4-((2-(((4-chlorophenyl)sulfonyl)amino)ethyl)thio)-2,6-difluorophenoxy)acetic acid, (1S-(1α,2β(Z),3α(S),5α))-7-(3-((cyclopentyl hydroxyacetyl)amino)-6,6-dimethylbicyclo (3.1.1)hept-2-yl)-5-heptenoic acid, (E)-3-(4-(1H-imidazol-1-ylmethyl)phenyl)-2-propenoic acid monohydrochloride, OKY 1555, OKY 1580, (E)-2-methyl-3-(4-(3-pyridinylmethyl)phenyl)-2-propenoic acid sodium salt, (±)-(5Z)-7-(3-endo-((phenylsulfonyl)amino)bicyclo (2.2.1)hept-2-exo-yl)heptenoic acid, 4-methoxy-N-N′-bis(3-pyridinylmethyl)-1,3-benzenedicarboxamide monohydrate, (E)-5-(((3-pyridinyl(3-(trifluoromethyl)phenyl)methylene)amino)oxy)-pentanoic acid, (1R)-(1α,2αZ),3β,4α))-7-(3-((phenylsulfonyl)amino)bicyclo(2.2.1)hept-2-yl)-5heptenoic acid, N7-(3chlorophenyl)-N2-((7-(((3-chlorophenyl)amino)sulfonyl)-3,4-dihydro-2(1H)-isoquinolinyl)sulfonyl)-3,4-dihydro-2,7(1H)-isoquinolinedisulfonamide, ((1)-(1α,2α(Z), 3α(1E,3R),4α))-7-(3-(3-cyclohexyl-3-hydroxy-1-propenyl)-7-oxabicyclo(2.2.1)hept-2-yl)-5-heptenoic acid, (1α,2α(Z), 3α(1E,3S,4R),4α)-7-(3-(3-hydroxy-4-phenyl-1-pentyl)-7-oxabicyclo(2.2.1)hept-2-yl)-5-heptenoic acid, ((1S)-(1α,2β(5Z),3β,4α))-7-(3-((2-(phenylamino)-carbonyl)hydrazino)methyl)-7-oxabicyclo(2.2.1)-hept-2-yl)-5-heptenoic acid, ((1)-(1α2α(Z),3α,4α))-7-(3-(((((1-oxoheptyl)amino)acetyl)amino)methy)-7-oxabicyclo(2.2.1)-hept-2-yl)-5-heptenoic acid, (1S-exo,exo))-2-((3-(4-(((4-cyclohexylbutyl)amino)carbonyl)-2-oxazolyl)-7-oxabicyclo(2.2.1)-hept-2-yl)methyl)-benzenepropanoic acid, 4-(1-(((4-chlorophenyl)sulfonyl)amino)methyl)-4-(3-pyridinyl)butyl)-benzenepropanoic acid, UK 34787, β,β-dimethyl-6-chloro-3-((4-chlorophenyl)methyl)-3H-imidazo(4,5-b)pyridine-2-butanoic acid, Y 20811, (3-((4-tert-butylthiazol-2-yl)methoxy)-5′-(3-(4-chlorobenzene sulfonyl)propyl-2′-(1H-tetrazol-5-ylmethoxy)benzanilide monosodium salt monohydrate, (±)-sodium(2-(4-(chlorophenyl sulfonylaminomethyl)indan-5-yl)acetate monohydrate, 4(Z)-6-(2S,4S,5R)-2-(1-methyl-1-(2-nitro-4-tolyloxy)ethyl)-4-(3-pyridyl)-1,3-dioxan-5-yl)hex-4-enoic acid or (4Z)-6-((2S,4S,5R)-2-(1-(2-cyano-4-methylphenoxy)-1-methylethyl)-4-(3-dioxan-5-yl)hex-4-enoic acid.

3. The method of claim 1, wherein the thromboxane inhibitor is a thromboxane A₂ receptor antagonist, a thromboxane synthase inhibitor or a dual acting thromboxane receptor antagonist and thromboxane synthase inhibitor.

4. The method of claim 1, wherein the patient is female.

5. The method of claim 1, wherein the patient is male.

6. The method of claim 1, wherein the patient is diabetic.

7. The method of claim 1, wherein the thromboxane inhibitor is administered orally, bucally, topically, by injection, by inhalation or by transurethral application.

8. The method of claim 7, wherein the thromboxane inhibitor is administered orally as a solid or a liquid dose.

9. The method of claim 7, wherein the thromboxane inhibitor is administered topically as a cream, a spray, a lotion, a gel, an ointment, an emulsion, a foam, a coating for a condom, or a liposome composition.

10. The method of claim 1, further comprising administering to the patient at least one vasoactive agent or a pharmaceutically acceptable salt thereof.

11. The method of claim 10, wherein the vasoactive agent is a potassium channel activator, a calcium channel blocker, an α-adrenergic receptor antagonist, a β-blocker, a phosphodiesterase inhibitor, adenosine, an ergot alkaloid, a vasoactive intestinal peptide, a prostaglandin, a dopamine agonist, an opioid antagonist, an endothelin antagonist or a mixture thereof.

12. A composition comprising at least one thromboxane inhibitor, or a pharmaceutically acceptable salt thereof, and at least one compound that donates, transfers or releases nitric oxide, elevates endogenous levels of endothelium-derived relaxing factor, stimulates endogenous synthesis of nitric oxide or is a substrate for nitric oxide synthase, or a pharmaceutically acceptable salt thereof.

13. The composition of claim 12, further comprising a pharmaceutically acceptable carrier.

14. The composition of claim 12, wherein the at least one thromboxane thromboxane inhibitor is 2-(acetyloxy)-benzoic acid, 4-hydroxy-(Z)-(2,4,5-trimethyl-3,6-dioxo-1,4-cyclohexa dien-1-yl)-benezeneheptanoic acid, (1α((Z),2β,5α)-(±)-7-(5-(((1,1′biphenyl)-4-yl)methoxy)-2-(4-morpholinyl)-3-oxocyclopentyl)-4-heptenoic acid, 3R-((4-fluorophenyl)sulfonyl)amino)-1,2,3,4-tetrahydro-9H-carbazole-9-propanoic acid, (4-(2-((phenylsulfonyl)amino)ethyl)phenoxy)-acetic acid, 4-(2-(((4-chlorophenyl)sulfonyl)amino)ethyl)-benzeneacetic acid, ((+)-1S-(1α,2α,3α,4α)-2-((3-(4-(n-pentylamino)carbonyl)-2-oxazolyl)-7-oxabicyclo(2.2.1)hept-2-yl)methyl)benzeneproponic acid, (8R,8′)-4,4′-dihydroxy-3,3′-dimethoxy-7-oxo-8,8′-neolignan, 3-methyl-2-(3-pyridyl)indole-1-octanoic acid, CGS 15435, 4-(((4-chlorophenyl)sulfonyl)amino)butyl)-3-pyridine heptanoic acid, ((E)-7-phenyl-7-(3-pyridinyl)-6-heptenoic acid), dazmegrel, 4-(2-(1H-imidazol-1-yl)ethoxy)benzoic acid, (E)-6-(4-2-(4-chlorobenzene sulphonyl amino)-ethyl)phenyl)-6-(3-pyridyl)-hex-5-enoic acid, 2-(2-(4-chlorophenoxy)-2-methyl-1-oxopropoxy)-3-pyridine carboxylic acid ethyl ester, (1α,2β(Z),3α,4α)-7-(3-((((phenylamino)carbonyl)hydrazono)methyl)bicyclo(2.2.1)hept-2-yl), 7-((1S,2S,3S,4R)-3-(1-(3-(phenylthio ureidoimino)ethyl)bicyclo(2.2.1)heptane-2-yl)-5-heptenoic acid, 1-(((5-(4-chlorophenyl)-2-furanyl)methylene)amino)-3-(4-(1-piperazinyl)butyl)-2,4-imidazolidine-dione, (E)-(±)-5-(((1-cyclohexyl-2-(1H-imidazol-1-yl)-3-phenyl propylidene)amino)oxy-pentanoic acid, 2,3-dihydro-5-(2-(1H-imidazol-1-yl)ethoxy)-indene-1-carboxylic acid, (5Z)-6-((2S,4R)-4-((4-chlorophenyl)sulfonyl)amino)-1-(3-pyridinylmethyl)-2-pyrrolidinyl)-5-hexenoic acid monohydrate, furegrelate, (1R-(1α(Z),2β,3β,5α))-(+)-7-(5-((1,1′-biphenyl)-4-ylmethoxy)-3-hydroxy-2-(1-piperidinyl)cyclopentyl)-4-++++heptonoic acid, (±)-(1α(Z),2β,5β)-6-((2-(hexahydro-1H-azepin-1-yl)-5-((4-(4-propyl-3-pyridinyl)phenyl) methoxy)cyclopentyl)oxy)-4-hexanoic acid, (5E)-6-(3-(2-((4-iodophenyl)sulfonyl)amino)ethyl)phenyl)-6-(3-pyridinyl)-5-hexenoic acid, (5E)-6-(3-(2-((4-iodophenyl)sulfonyl)amino)-1,1-dimethylethyl)phenyl)-6-(3-pyridinyl)-5-hexenoic acid, 2-(6-carboxyhexyl)-3-n-hexylcyclohexylamine, (E)-7-phenyl-7-(3-pyridinyl)-6-heptenoic acid, 5(Z)-7-(2,2-dimethyl-4-(2-hydroxyphenyl)-1,3-dioxan-cis-5-yl)heptenoic acid, (Z)-(2α,4α,5α)-6-(2-(2-chlorophenyl)-4-(2-hydroxyphenyl)-1,3-dioxan-5-yl)-4-hexenoic acid, 3-(4-(((4-chlorophenyl)sulfonyl)amino)butyl)-6-(1-methylethyl)-1-azulenesulfonic acid monosodium salt, (E)-11-(2-(5,6-dimethyl-1H-benzimidazol-1-yl)-6,11-dihydro-dibenz(b,e)oxepin-2-carboxylic acid sodium salt, sodium (E)-11-(2-(5,6-dimethyl-1-benzimidazolyl)ethylidene)-6,11-dihydrodibenz (b,e)oxepin-2-carboxylate monohydrate, 4-methyl-benzenesulfonate-N-((phenylmethoxy)carbonyl)-serine ethyl ester, dibenzo(b,f) thiepin-3-methanol-5,5-dioxide, (−)-6,8-difloro-2,3,4,9-tetrahydro-9-((4-(methylsulfonyl)phenyl)methyl)-1H-carbazole-1-acetic acid, (5,5-dioxide-dibenzo(b,f)thiepin-3-carboxylic acid, 3-methyl-1-((4-chlorophenyl)methyl)-5-fluoro-α,α,-1H-indole-2-propanoic acid, midazogrel, (4-((2-(((4-chlorophenyl)sulfonyl)amino)ethyl)thio)-2,6-difluorophenoxy)acetic acid, (1S-(1α,2β(Z),3α(S),5α))-7-(3-((cyclopentyl hydroxyacetyl)amino)-6,6-dimethylbicyclo(3.1.1)hept-2-yl)-5-heptenoic acid, (E)-3-(4-(1H-imidazol-1-ylmethyl)phenyl)-2-propenoic acid monohydrochloride, OKY 1555, OKY 1580, (E)-2-methyl-3-(4-(3-pyridinylmethyl)phenyl)-2-propenoic acid sodium salt, (±)-(5Z)-7-(3-endo-((phenylsulfonyl)amino)bicyclo(2.2.1)hept-2-exo-yl)heptenoic acid, 4-methoxy-N-N′-bis(3-pyridinylmethyl)-1,3-benzenedicarboxamide monohydrate, (E)-5-(((3-pyridinyl(3-(trifluoromethyl)phenyl)methylene)amino)oxy)-pentanoic acid, (1R)-(1α,2α(Z),3β,4α))-7-(3-((phenylsulfonyl)amino)bicyclo(2.2.1)hept-2-yl)-5-heptenoic acid, N7-(3-chlorophenyl)-N2-((7-(((3-chlorophenyl)amino)sulfonyl)-3,4-dihydro-2(1H)-isoquinolinyl)sulfonyl)-3,4-dihydro-2,7(1H)-isoquinolinedisulfonamide, ((1S)-(1α,2α(Z), 3α(1E,3R),4α)) -7-(3-(3-cyclohexyl-3-hydroxy-1-propenyl)-7-oxabicyclo(2.2.1)hept-2-yl)-5-heptenoic acid, (1α,2α(Z), 3α(1E,3S,4R),4α)-7-(3-(3-hydroxy-4-phenyl-1-pentyl)-7-oxabicyclo(2.2.1)hept-2-yl)-5-heptenoic acid, ((1S)-(1α,2β(5Z),3β4α))-7-(3-((2-((phenylamino)-carbonyl)hydrazino)methyl)-7-oxabicyclo(2.2.1)-hept-2-yl)-5-heptenoic acid, ((1S)-(1α2α(Z), 3α,4α))-7-(3-(((((1-oxoheptyl)amino)acetyl)amino)methy)-7-oxabicyclo(2.2.1)-hept-2-yl)-5-heptenoic acid, (1S-exo,exo))-2-((3-(4-(((4-cyclohexylbutyl)amino)carbonyl)-2-oxazolyl)-7-oxabicyclo(2.2.1)-hept-2-yl)methyl)-benzenepropanoic acid, 4-(1-(((4-chlorophenyl)sulfonyl)amino)methyl)-4-(3-pyridinyl)butyl)-benzenepropanoic acid, UK 34787, β,β-dimethyl-6-chloro-3-((4-chlorophenyl)methyl)-3H-imidazo(4,5-b)pyridine-2-acid, Y 20811, (3-((4-tert-butylthiazol-2-yl)methoxy)-5′-(3-(4-chlorobenzene sulfonyl)propyl-2′-(1H-tetrazol-5-ylmethoxy)benzanilide monosodium salt monohydrate, (±)-sodium(2-(4-(chlorophenylsulfonylaminomethyl)indan-5-yl)acetate monohydrate, 4(Z)-6-(2S,4S,5R)-2-(1-methyl-1-(2-nitro-4-tolyloxy)ethyl)-4-(3-pyridyl)-1,3-dioxan-5-yl)hex-4-enoic acid or (4Z)-6-((2S,4S,5R)-2-(1-(2-cyano-4-methylphenoxy)-1-methylethyl)4-(3-pyridyl)-1,3-dioxan-5-yl)hex-4-enoic acid.

15. The composition of claim 12, wherein the thromboxane inhibitor is a thromboxane A₂ receptor antagonist, a thromboxane synthase inhibitor or a dual acting thromboxane receptor antagonist and thromboxane synthase inhibitor.

16. The composition of claim 12, wherein the at least one compound that donates, transfers or releases nitric oxide, elevates endogenous levels of endothelium-derived relaxing factor, stimulates endogenous synthesis of nitric oxide or is a substrate for nitric oxide synthase is an S-nitrosothiol.

17. The composition of claim 16, wherein the S-nitrosothiol is S-nitroso-N-acetylcysteine, S-nitroso-captopril, S-nitroso-N-acetylpenicillamine, S-nitroso-homnocysteine, S-nitroso-cysteine or S-nitroso-glutathione.

18. The composition of claim 16, wherein the S-nitrosothiol is:
(i) HS(C(Rₑ)(R_{f}))ₘSNO;
(ii) ONS(C(Rₑ)(R_{f}))ₘRₑ; and
(iii) H₂N—CH(CO₂H)—(CH₂)ₘ—C(O)NH—CH(CH₂SNO)—C(O)NH—CH₂—CO₂H;
wherein m is an integer from 2 to 20; Rₑ and R_{f} are each independently a hydrogen, an alkyl, a cycloalkoxy, a halogen, a hydroxy, an hydroxyalkyl, an alkoxyalkyl, an arylheterocyclic ring, an alkylaryl, a cycloalkylalkyl, a heterocyclicalkyl, an alkoxy, a haloalkoxy, an amino, an alkylamino, a dialkylamino, an arylamino, a diarylamino, an alkylarylamino, an alkoxyhaloalkyl, a haloalkoxy, a sulfonic acid, a sulfonic ester, an alkylsulfonic acid, an arylsulfonic acid, an arylalkoxy, an alkylthio, an arylthio, a cycloalkylthio, a cycloalkenyl, a cyano, an aminoalkyl, an aminoaryl, an aryl, an arylalkyl, an alkylaryl, a carboxamido, a alkylcarboxamido, an arylcarboxamido, an amidyl, a carboxyl, a carbamoyl, a carbamate, an alkylcarboxylic acid, an arylcarboxylic acid, an alkylcarbonyl, an arylcarbonyl, an ester, a carboxylic ester, an alkylcarboxylic ester, an arylcarboxylic ester, a haloalkoxy, a sulfonamido, an alkylsulfonamido, an arylsulfonamido, a sulfonic ester, a urea, a phosphoryl, a nitro, —T—Q , or —(C(Rₑ)(R_{f})ₖ—T—Q, or Rₑ and R_{f} taken together with the carbons to which they are attached form a carbonyl, a methanthial, a heterocyclic ring, a cycloalkyl group or a bridged cycloalkyl group; Q is —NO or —NO₂; and T is independently a covalent bond, a carbonyl, an oxygen, —S(O)ₒ— or —N(Rₐ)Rᵢ—, wherein o is an integer from 0 to 2, Rₐ is a lone pair of electrons, a hydrogen or an alkyl group; Rᵢ is a hydrogen, an alkyl, an aryl, an alkylcarboxylic acid, an aryl carboxylic acid, an alkylcarboxylic ester, an arylcarboxylic ester, an alkylcarboxamido, an arylcarboxamido, an alkylaryl, an alkylsulfinyl, an alkylsulfonyl, an arylsulfinyl, an arylsulfonyl, a sulfonamido, a carboxamido, a carboxylic ester, an amino alkyl, an amino aryl, —CH₂—C(T—Q)(Rₑ)(R_{f}), or —(N₂O₂—)⁻.M⁺, wherein M⁺ is an organic or inorganic cation; with the proviso that when Rᵢ is —CH₂—C(T—Q)(Rₑ)(R_{f}) or —(N₂O₂—).M⁺; then “—T—Q” can be a hydrogen, an alkyl group, an alkoxyalkyl group, an aminoalkyl group, a hydroxy group or an aryl group.

19. The composition of claim 12, wherein the at least one compound that donates, transfers or releases nitric oxide, elevates endogenous levels of endothelium-derived relaxing factor, stimulates endogenous synthesis of nitric oxide or is a substrate for nitric oxide synthase is:
(i) a compound that comprises at least one ON—O—, ON—N— or ON—C— group;
(ii) a compound that comprises at least one O₂N—O—, O₂N—N—, O₂N—S— or —O₂N—C— group;
(iii) a N-oxo-N-nitrosoamine having the formula: R¹R²—N(O—M⁺)—NO, wherein R¹ and R² are each independently a polypeptide, an amino acid, a sugar, an oligonucleotide, a straight or branched, saturated or unsaturated, aliphatic or aromatic, substituted or unsubstituted hydrocarbon, or a heterocyclic group, and M⁺ is an organic or inorganic cation.

20. The composition of claim 19, wherein the compound comprising at least one ON—O—, ON—N— or ON—C— group is an ON—O-polypeptide, an ON—N-polypeptide, an ON—C-polypeptide, an ON—O-amino acid, an ON—N-amino acid, an ON—C-amino acid, an ON—O-sugar, an ON—N-sugar, an ON—C-sugar, an ON—O-oligonucleotide, an ON—N-oligonucleotide, an ON—C-oligonucleotide, a straight or branched, saturated or unsaturated, substituted or unsubstituted, aliphatic or aromatic ON—O-hydrocarbon, a straight or branched, saturated or unsaturated, substituted or unsubstituted, aliphatic or aromatic ON—N-hydrocarbon, a straight or branched, saturated or unsaturated, substituted or unsubstituted, aliphatic or aromatic ON—C-hydrocarbon, an ON—O-heterocyclic compound, an ON—N-heterocyclic compound or a ON—C-heterocyclic compound.

21. The composition of claim 19, wherein compound comprising at least one O₂N—O—, O₂N—N—, O₂N—S— or O₂N—C— group is an O₂N—O-polypeptide, an O₂N-N-polypeptide, an O₂N—S-polypeptide, an O₂N—C-polypeptide, an O₂N—O-amino acid, O₂N—N-amino acid, O₂N—S-amino acid, an O₂N—C-amino acid, an O₂N—O-sugar, an O₂N—N-sugar, O₂N—S-sugar, an O₂N—C-sugar, an O₂N—O-oligonucleotide, an O₂N—N-oligonucleotide, an O₂N—S-oligonucleotide, an O₂N—C-oligonucleotide, a straight or branched, saturated or unsaturated, aliphatic or aromatic, substituted or unsubstituted O₂N—O-hydrocarbon, a straight or branched, saturated or unsaturated, aliphatic or aromatic, substituted or unsubstituted O₂N—N-hydrocarbon, a straight or branched, saturated or unsaturated, aliphatic or aromatic, substituted or unsubstituted O₂N—S-hydrocarbon, a straight or branched, saturated or unsaturated, aliphatic or aromatic, substituted or unsubstituted O₂N—C-hydrocarbon, an O₂N—O-heterocyclic compound, an O₂N—N-heterocyclic compound, an O₂N—S-heterocyclic compound or an O₂N—C-heterocyclic compound.

22. The composition of claim 12, wherein the at least one compound that donates, transfers or releases nitric oxide, elevates endogenous levels of endothelium-derived relaxing factor, stimulates endogenous synthesis of nitric oxide or is a substrate for nitric oxide synthase, is L-arginine, L-homoarginine, N-hydroxy-L-arginine, nitrosated L-arginine, nitrosylated L-arginine, nitrosated N-hydroxy-L-arginine, nitrosylated N-hydroxy-L-arginine, citrulline, ornithine, glutamine, lysine, polypeptides comprising at least one of these amino acids or inhibitors of the enzyme arginase.

23. The composition of claim 12, wherein the at least one compound that donates, transfers or releases nitric oxide, elevates endogenous levels of endothelium-derived relaxing factor, stimulates endogenous synthesis of nitric oxide or is a substrate for nitric oxide synthase is a NONOate.

24. The composition of claim 12, wherein the composition is administered orally, bucally, topically, by injection, by inhalation or by transurethral application.

25. The composition of claim 24, wherein the composition is administered orally as a solid or a liquid dose.

26. The composition of claim 24, wherein the composition is administered topically as a cream, a spray, a lotion, a gel, an ointment, an emulsion, a foam, a coating for a condom, or a liposome composition.

27. A method for treating a sexual dysfunction in a patient in need thereof comprising administering to the patient a therapeutically effective amount of the composition of claim 12.

28. The method of claim 27, wherein the patient is female.

29. The method of claim 27, wherein the patient is male.

30. The method of claim 27, wherein the patient is diabetic.

31. The method of claim 27, wherein the composition is administered orally, bucally, topically, by injection, by inhalation or by transurethral application.

32. The method of claim 31, wherein the composition is administered orally as a solid or a liquid dose.

33. The method of claim 31, wherein the composition is administered topically as a cream, a spray, a lotion, a gel, an ointment, an emulsion, a foam, a coating for a condom, or a liposome composition.

34. A method for treating or preventing an ischemic heart disorder, a myocardial infarction, an angina pectoris, a stroke, a migraine, a cerebral hemorrhage, a cardiac fatality, a transient ischaemic attack, a complication following an organ transplant, a coronary artery bypass, an angioplasty, an endarterectomy, atherosclerosis, a pulmonary embolism, bronchial asthma, bronchitis, pneumonia, circulatory shock of various organs, nephritis, a graft rejection, a cancerous metastases, a pregnancy-induced hypertension, a preeclampsia, an eclampsia, a thrombotic or thromboembolic disorder, an intrauterine growth, a gastrointestinal disorder, a renal disease or disorder, a disorder resulting from elevated uric acid levels, a dysmenorrhea, or for inhibiting platelet aggregation or platelet adhesion or relaxing smooth muscles in a patient in need thereof comprising administering to the patient a therapeutically effective amount of the composition of claim 12.

35. The composition of claim 12, further comprising at least one therapeutic agent or a pharmaceutically acceptable salt thereof.

36. The composition of claim 35, wherein the therapeutic agent is a vasoactive agent, a nonsteroidal antiinflammatory compound, a selective cyclooxygenase-2 inhibitor, an anticoagulant, an angiotensin converting enzyme inhibitor, an angiotensin II receptor antagonist, a renin inhibitor or a mixture thereof.

37. The composition of claim 36, wherein the vasoactive agent is a potassium channel activator, a calcium channel blocker, an α-adrenergic receptor antagonist, a β-blocker, a phosphodiesterase inhibitor, adenosine, an ergot alkaloid, a vasoactive intestinal peptide, a prostaglandin, a dopamine agonist, an opioid antagonist, an endothelin antagonist or a mixture thereof.

38. A method for treating a sexual dysfunction in a patient in need thereof comprising administering to the patient a therapeutically effective amount of the composition of claim 37.

39. The method of claim 38, wherein the patient is female.

40. The method of claim 38, wherein the patient is male.

41. The method of claim 38, wherein the patient is diabetic.

42. The method of claim 38, wherein the composition is administered orally, bucally, topically, by injection, by inhalation or by transurethral application.

43. The method of claim 42, wherein the composition is administered orally as a solid or a liquid dose.

44. The method of claim 42, wherein the composition is administered topically as a cream, a spray, a lotion, a gel, an ointment, an emulsion, a foam, a coating for a condom, or a liposome composition.

45. A method for treating or preventing an ischemic heart disorder, a myocardial infarction, an angina pectoris, a stroke, a migraine, a cerebral hemorrhage, a cardiac fatality, a transient ischemic attack, a complication following an organ transplant, a coronary artery bypass, an angioplasty, an endarterectomy, atherosclerosis, a pulmonary embolism, bronchial asthma, bronchitis, pneumonia, circulatory shock of various organs, nephritis, a graft rejection, a cancerous metastases, a pregnancy-induced hypertension, a preeclampsia, an eclampsia, a thrombotic or thromboembolic disorder, an intrauterine growth, a gastrointestinal disorder, a renal disease or disorder, a disorder resulting from elevated uric acid levels, a dysmenorrhea, inhibiting platelet aggregation or platelet adhesion or relaxing smooth muscles in a patient in need thereof comprising administering to the patient a therapeutically effective amount of the composition of claim 35.

46. A kit comprising at least one thromboxane inhibitor and at least one compound that donates, transfers or releases nitric oxide, elevates endogenous levels of endothelium-derived relaxing factor, stimulates endogenous synthesis of nitric oxide or is a substrate for nitric oxide synthase, or a pharmaceutically acceptable salt thereof.

47. The kit of claim 46, wherein the at least one thromboxane inhibitor is 2-(acetyloxy)-benzoic acid, 4-hydroxy-(Z)-(2,4,5-trimethyl-3,6-dioxo-1,4-cyclohexa dien-1-yl)-benzeneheptanoic acid, (1α(Z),2β,5α)-(±)-7-(5-(((1,1′-biphenyl)-4-yl)methoxy)-2-(4-morpholinyl)-3-oxocyclopentyl)-4-heptenoic acid, 3R-((4-fluorophenyl)sulfonyl)amino-1,2,3,4-tetrahydro-9H-carbazole-9-propanoic acid, (4-(2-((phenylsulfonyl)amino)ethyl)phenoxy)-acetic acid, 4-(2-(((4-chlorophenyl)sulfonyl)amino)ethyl)-benzeneacetic acid, ((+)-1S-(1α,2α3α,4α)-2-((3-(4-(n-pentylamino)carbonyl)-2-oxazolyl)-7-oxabicyclo(2.2.1hept-2-yl)methyl)benzeneproponic acid, (8R,8′S)-4,4′-dihydroxy-3,3′-dimethoxy-7-oxo-8,8′-neolignan, 3-methyl-2-(3-pyridyl)indole-1-octanoic acid, CGS 15435, 4-(((4-chlorophenyl)sulfonyl)amino)butyl)-3-pyridine heptanoic acid, ((E)-7-phenyl-7-(3-pyridinyl)-6-heptenoic acid), dazmegrel, 4-(2-(1H-imidazol-1-yl)ethoxy)benzoic acid, (E)-6-(4-2-(4-chlorobenzene sulphonyl amino)-ethyl)phenyl)-6-(3-pyridyl)-hex-5-enoic acid, 2-(2-(4-chlorophenoxy)-2-methyl-1-oxopropoxy)-3-pyridinecarboxylic acid ethyl ester, (1α,2β(Z),3α,4α)-7-(3-((((phenylamino)carbonyl)hydrazono)methyl)bicyclo(2.2.1)hept-2-yl), 7-((1S,2S,3S,4R)-3-(1-(3-(phenylthio ureidoimino)ethyl)-bicyclo(2.2.1)heptane-2-yl)-5-heptenoic acid, 1-(((5-(4-chlorophenyl)-2-furanyl)methylene)amino)-3-(4-(1-piperazinyl)butyl)-2,4-imidazolidine-dione, (E)-(±)-5-(((1-cyclohexyl-2-(1H-imidazol-1-yl)-3-phenyl propylidene)amino)oxy-pentanoic acid, 2,3-dihydro-5-(2-(1H-imidazol-1-yl)ethoxy)-indene-1-carboxylic acid, (5Z)-6-((2S,4R)-4-((4-chlorophenyl)sulfonyl)amino)-1-(3-pyridinyl methyl)-2-pyrrolidinyl)-5-hexenoic acid monohydrate, furegrelate, (1R-(1α(Z),2β,3β,5α))-(+)-7-(5-((1,1′-biphenyl)-4-ylmethoxy)-3-hydroxy-2-(1-piperidinyl)cyclopentyl)-4-++++heptonoic acid, (±)-(1α(Z),2β,5β)-6-((2-(hexahydro-1H-azepin-1-yl)-5-((4-(4-propyl-3-pyridinyl)phenyl)methoxy)cyclopentyl)oxy)-4-hexanoic acid, (5E)-6-(3-(2-((4-iodophenyl)sulfonyl)amino)ethyl)phenyl)-6-(3-pyridinyl)-5-hexenoic acid, (5E)-6-(3-(2-((4-iodophenyl)sulfonyl)amino)-1,1-dimethylethyl)phenyl)-6-(3-pyridinyl)-5-hexenoic acid, 2-(6-carboxyhexyl)-3-n-hexylcyclohexylamine, (E)-7-phenyl-7-(3-pyridinyl)-6-heptenoic acid, 5(Z)-7-(2,2-dimethyl-4-(2-hydroxyphenyl)-1,3-dioxan-cis-5-yl)heptenoic acid, (Z)-(2α4α,5α)-6-(2-(2-chlorophenyl)-4-(2-hydroxyphenyl)-1,3-dioxan-5-yl)-4-hexenoic acid, 3-(4-(((4-chlorophenyl)sulfonyl)amino)butyl)-6-(1-methylethyl)-1-azulenesulfonic acid monosodium salt, (E)-11-(2-(5,6-dimethyl-1H-benzimidazol-1-yl)-6,11-dihydro-dibenz(b,e)oxepin-2-carboxylic acid sodium salt, sodium (E)-11-(2-(5,6-dimethyl-1-benzimidazolyl)ethylidene)-6,11-dihydrodibenz (b,e)oxepin-2-carboxylate monohydrate, 4-methyl-benzenesulfonate-N-((phenylmethoxy)carbonyl)-serine ethyl ester, dibenzo (b,f)thiepin-3-methanol-5,5-dioxide, (−)-6,8-difloro-2,3,4,9-tetrahydro-9-((4-(methylsulfonyl)phenyl)methyl)-1H-carbazole-1-acetic acid, (5,5-dioxide-dibenzo(b,f) thiepin-3-carboxylic acid, 3-methyl-1-((4-chlorophenyl)methyl)-5-fluoro-α,α,-1H-indole-2-propanoic acid, midazogrel, (4-((2-(((4-chlorophenyl)sulfonyl)amino)ethyl)thio)-2,6-difluorophenoxy) acetic acid, (1S-(1α,2β(Z),3α(S),5α))-7-(3-((cyclopentylhydroxyacetyl)amino)-6,6-dimethylbicyclo (3.1.1)hept-2-yl)-5-heptenoic acid, (E)-3-(4-(1H-imidazol-1-ylmethyl)phenyl)-2-propenoic acid monohydrochloride, OKY 1555, OKY 1580, (E)-2-methyl-3-(4-(3-pyridinylmethyl)phenyl)-2-propenoic acid sodium salt, (±)-(5Z)-7-(3-endo ((phenylsulfonyl)amino)bicyclo (2.2.1)hept-2-exo-yl)heptenoic acid, 4-methoxy-N-N′-bis(3 pyridinylmethyl)-1,3-benzenedicarboxamide monohydrate, (E)-5-(((3-pyridinyl(3-(trifluoromethyl)phenyl)methylene)amino)oxy)-pentanoic acid, (1R)-(1α,2α(Z),3β,4α))-7-(3-((phenylsulfonyl)amino)bicyclo(2.2.1)hept-2-yl)-5-heptenoic acid, N7-(3-chlorophenyl)-N2-((7-(((3-chlorophenyl)amino)sulfonyl)-3,4-dihydro-2(1H)-isoquinolinyl)sulfonyl)-3,4-dihydro-2,7(1H)-isoquinolinedisulfonamide, ((1S)-(1α,2α(Z), 3α(1E,3R),4α))-7-(3-(3-cyclohexyl-3-hydroxy-1-propenyl)-7-oxabicyclo(2.2.1) hept-2-yl)-5-heptenoic acid, (1α,2α(Z), 3α(1E,3S,4R),4α)-7-(3-(3-hydroxy-4-phenyl-1-pentyl)-7-oxabicyclo(2.2.1)hept-2-yl)-5-heptenoic acid, ((1S)-(1α,2β(5Z),3β,4α))-7-(3-((2-((phenylamino)-carbonyl)hydrazino)methyl)-7-oxabicyclo(2.2.1)-hept-2-yl)-5-heptenoic acid, ((1S)-(1α,2α(Z), 3α,4α))-7-(3-(((((1-oxoheptyl)amino)acetyl)amino)methy)-7-oxabicyclo(2.2.1)-hept-2-yl)-5-heptenoic acid, (1S-exo,exo))-2-((3-(4-(((4-cyclohexylbutyl)amino)carbonyl)-2-oxazolyl)-7-oxabicyclo(2.2.1-hept-2-yl)methyl)-benzenepropanoic acid, 4-(1-(((4-chlorophenyl)sulfonyl)amino)methyl)-4-(3-pyridinyl)butyl)-benzenepropanoic acid, UK 34787, β,β-dimethyl-6-chloro-3-((4-chlorophenyl)methyl)-3H-imidazo(4,5-b)pyridine-2-butanoic acid, Y 20811, (3-((4-tert-butylthiazol-2-yl)methoxy)-5′-(3-(4-chlorobenzene sulfonyl)propyl-2′-(1H-tetrazol-5-ylmethoxy)benzanilide monosodium salt monohydrate, (±)-sodium(2-(4-(chlorophenyl sulfonylaminomethyl)indan-5-yl)acetate monohydrate, 4(Z)-6-(2S,4S,5R)-2-(1-methyl-1-(2-nitro-4-tolyloxy)ethyl)-4-(3-pyridyl)-1,3-dioxan-5-yl)hex-4-enoic acid or (4Z)-6-((2S,4S,5R)-2-(1-(2-cyano-4-methylphenoxy)-1-methylethyl)-4-(3-pyridyl)-1,3-dioxan-5-yl)hex-4-enoic acid.

48. The kit of claim 46, wherein the thromboxane inhibitor is a thromboxane A₂ receptor antagonist, a thromboxane synthase inhibitor or a dual acting thromboxane receptor antagonist and thromboxane synthase inhibitor.

49. The kit of claim 46, wherein the at least one compound that donates, transfers or releases nitric oxide, elevates endogenous levels of endothelium-derived relaxing factor, stimulates endogenous synthesis of nitric oxide or is a substrate for nitric oxide synthase is an S-nitrosothiol.

50. The kit of claim 49, wherein the S-nitrosothiol is S-nitroso-N-acetylcysteine, S-nitroso-captopril, S-nitroso-N-acetylpenicillamine, S-nitroso-homocysteine, S-nitrosocysteine or S-nitroso-glutathione.

51. The kit of claim 49, wherein the S-nitrosothiol is:
(i) HS(C(Rₑ)(R_{f})ₘSNO;
(ii) ONS(C(Rₑ)(R_{f}))ₘRₑ; and
(iii) H₂N—CH(CO₂H)—(CH₂)ₘ—C(O)NH—CH(CH₂SNO)—C(O)NH—CH₂—CO₂H;
wherein m is an integer from 2 to 20; Rₑ and R_{f} are each independently a hydrogen, an alkyl, a cycloalkoxy, a halogen, a hydroxy, an hydroxyalkyl, an alkoxyalkyl, an arylheterocyclic ring, an alkylaryl, a cycloalkylalkyl, a heterocyclicalkyl, an alkoxy, a haloalkoxy, an amino, an alkylamino, a dialkylamino, an arylamino, a diarylamino, an alkylarylamino, an alkoxyhaloalkyl, a haloalkoxy, a sulfonic acid, a sulfonic ester, an alkylsulfonic acid, an arylsulfonic acid, an arylalkoxy, an alkylthio, an arylthio, a cycloalkylthio, a cycloalkenyl, a cyano, an aminoalkyl, an aminoaryl, an aryl, an arylalkyl, an alkylaryl, a carboxamido, a alkylcarboxamido, an arylcarboxamido, an amidyl, a carboxyl, a carbamoyl, a carbamate, an alkylcarboxylic acid, an arylcarboxylic acid, an alkylcarbonyl, an arylcarbonyl, an ester, a carboxylic ester, an alkylcarboxylic ester, an arylcarboxylic ester, a haloalkoxy, a sulfonamido, an alkylsulfonamido, an arylsulfonamido, a sulfonic ester, a urea, a phosphoryl, a nitro, —T—Q , or —(C(Rₑ)(R_{f}))ₖ—T—Q, or Rₑ and R_{f} taken together with the carbons to which they are attached form a carbonyl, a methanthial, a heterocyclic ring, a cycloalkyl group or a bridged cycloalkyl group; Q is —NO or —NO₂; and T is independently a covalent bond, a carbonyl, an oxygen, —S(O)ₒ— or —N(Rₐ)Rᵢ—, wherein o is an integer from 0 to 2, Rₐ is a lone pair of electrons, a hydrogen or an alkyl group; Rᵢ is a hydrogen, an alkyl, an aryl, an alkylcarboxylic acid, an aryl carboxylic acid, an alkylcarboxylic ester, an arylcarboxylic ester, an alkylcarboxamido, an arylcarboxamido, an alkylaryl, an alkylsulfinyl, an alkylsulfonyl, an arylsulfinyl, an arylsulfonyl, a sulfonamido, a carboxamido, a carboxylic ester, an amino alkyl, an amino aryl, —CH₂—C(T—Q)(Rₑ)(R_{f}), or —(N₂O₂—)⁻.M⁺, wherein M⁺ is an organic or inorganic cation; with the proviso that when Rᵢ is —CH₂—C(T—Q)(Rₑ)(R_{f}) or —(N₂O₂—).M⁺; then “—T—Q” can be a hydrogen, an alkyl group, an alkoxyalkyl group, an aminoalkyl group, a hydroxy group or an aryl group.

52. The kit of claim 46, wherein the at least one compound that donates, transfers or releases nitric oxide, elevates endogenous levels of endothelium-derived relaxing factor, stimulates endogenous synthesis of nitric oxide or is a substrate for nitric oxide synthase is:
(i) a compound that comprises at least one ON—O—, ON—N— or ON—C— group;
(ii) a compound that comprises at least one O₂N—O—, O₂N—N—, O₂N—S— or —O₂N—C— group;
(iii) a N-oxo-N-nitrosoamine having the formula: R¹R²—N(O—M⁺)—NO, wherein R¹ and R² are each independently a polypeptide, an amino acid, a sugar, an oligonucleotide, a straight or branched, saturated or unsaturated, aliphatic or aromatic, substituted or unsubstituted hydrocarbon, or a heterocyclic group, and M⁺ is an organic or inorganic cation.

53. The kit of claim 52, wherein the compound comprising at least one ON—O—, ON—N— or ON—C— group is an ON—-polypeptide, an ON—N-polypeptide, an ON—C-polypeptide, an ON—O-amino acid, an ON—N-amino acid, an ON—C-amino acid, an ON—O-sugar, an ON—N-sugar, an ON—C-sugar, an ON—O-oligonucleotide, an ON—N-oligonucleotide, an ON—C-oligonucleotide, a straight or branched, saturated or unsaturated, substituted or unsubstituted, aliphatic or aromatic ON—O-hydrocarbon, a straight or branched, saturated or unsaturated, substituted or unsubstituted, aliphatic or aromatic ON—N-hydrocarbon, a straight or branched, saturated or unsaturated, substituted or unsubstituted, aliphatic or aromatic ON—C-hydrocarbon, an ON—O-heterocyclic compound, an ON—N-heterocyclic compound or a ON—C-heterocyclic compound.

54. The kit of claim 52, wherein compound comprising at least one O₂N—O—, O₂N—N—, O₂N—S— or O₂N—C— group is an O₂N—O-polypeptide, an O₂N—N-polypeptide, an O₂N—S-polypeptide, an O₂N—C-polypeptide, an O₂N—O-amino acid, O₂N—N-amino acid, O₂N—S-amino acid, an O₂N—C-amino acid, an O₂N—O-sugar, an O₂N—N-sugar, O₂N—S-sugar, an O₂N—C-sugar, an O₂N—O-oligonucleotide, an O₂N—N-oligonucleotide, an O₂N—S-oligonucleotide, an O₂N—C-oligonucleotide, a straight or branched, saturated or unsaturated, aliphatic or aromatic, substituted or unsubstituted O₂N—O-hydrocarbon, a straight or branched, saturated or unsaturated, aliphatic or aromatic, substituted or unsubstituted O₂N—N-hydrocarbon, a straight or branched, saturated or unsaturated, aliphatic or aromatic, substituted or unsubstituted O₂N—S-hydrocarbon, a straight or branched, saturated or unsaturated, aliphatic or aromatic, substituted or unsubstituted O₂N—C-hydrocarbon, an O₂N—O-heterocyclic compound, an O₂N—N-heterocyclic compound, an O₂N—S-heterocyclic compound or an O₂N—C-heterocyclic compound.

55. The kit of claim 46, wherein the at least one compound that donates, transfers or releases nitric oxide, elevates endogenous levels of endothelium-derived relaxing factor, stimulates endogenous synthesis of nitric oxide or is a substrate for nitric oxide synthase, is L-arginine, L-homoarginine, N-hydroxy-L-arginine, nitrosated L-arginine, nitrosylated L-arginine, nitrosated N-hydroxy-L-arginine, nitrosylated N-hydroxy-L-arginine, citrulline, ornithine, glutamine, lysine, polypeptides comprising at least one of these amino acids or inhibitors of the enzyme arginase.

56. The kit of claim 46, wherein the at least one compound that donates, transfers or releases nitric oxide, elevates endogenous levels of endothelium-derived relaxing factor, stimulates endogenous synthesis of nitric oxide or is a substrate for nitric oxide synthase is a NONOate.

57. The kit of claim 46, further comprising at least one therapeutic agent.

58. The kit of claim 57, wherein the therapeutic agent is a vasoactive agent, a nonsteroidal antiinflammatory compound, a selective cyclooxygenase-2 inhibitor, an anticoagulant, an angiotensin converting enzyme inhibitor, an angiotensin II receptor antagonist, a renin inhibitor or a mixture thereof.

59. The kit of claim 58, wherein the vasoactive agent is a potassium channel activator, a calcium channel blocker, an α-adrenergic receptor antagonist, a β-blocker, a phosphodiesterase inhibitor, adenosine, an ergot alkaloid, a vasoactive intestinal peptide, a prostaglandin, a dopamine agonist, an opioid antagonist, an endothelin antagonist or a mixture thereof.

60. The kit of claim 46, wherein the thromboxane inhibitor and the compound that donates, transfers or releases nitric oxide, elevates endogenous levels of endothelium-derived relaxing factor, stimulates endogenous synthesis of nitric oxide or is a substrate for nitric oxide synthase are separate components in the kit.

61. The kit of claim 46, wherein the thromboxane inhibitor and the compound that donates, transfers or releases nitric oxide, elevates endogenous levels of endothelium-derived relaxing factor, stimulates endogenous synthesis of nitric oxide or is a substrate for nitric oxide synthase are in the form of a composition in the kit.

62. A method for treating a sexual dysfunction in a patient in need thereof comprising administering to the patient at least one thromboxane inhibitor and at least one compound that donates, transfers or releases nitric oxide, elevates endogenous levels of endothelium-derived relaxing factor, stimulates endogenous synthesis of nitric oxide or is a substrate for nitric oxide synthase.

63. The method of claim 62, further comprising administering to the patient at least one vasoactive agent.

64. A method for treating or method for treating or preventing an ischemic heart disorder, a myocardial infarction, an angina pectoris, a stroke, a migraine, a cerebral hemorrhage, a cardiac fatality, a transient ischaemic attack, a complication following an organ transplant, a coronary artery bypass, an angioplasty, an endarterectomy, atherosclerosis, a pulmonary embolism, bronchial asthma, bronchitis, pneumonia, circulatory shock of various organs, nephritis, a graft rejection, a cancerous metastases, a pregnancy-induced hypertension, a preeclampsia, an eclampsia, a thrombotic or thromboembolic disorder, an intrauterine growth, a gastrointestinal disorder, a renal disease or disorder, a disorder resulting from elevated uric acid levels, a dysmenorrhea, inhibiting platelet aggregation or platelet adhesion or relaxing smooth muscles in a patient in need thereof comprising administering to the patient at least one thromboxane inhibitor and at least one compound that donates, transfers or releases nitric oxide, elevates endogenous levels of endothelium-derived relaxing factor, stimulates endogenous synthesis of nitric oxide or is a substrate for nitric oxide synthase.

65. The method of claim 63, further comprising administering to the patient at least one therapeutic agent.

66. The method of claim 62 or 64, wherein the at least one thromboxane inhibitor and the at least one compound that donates, transfers or releases nitric oxide, elevates endogenous levels of endothelium-derived relaxing factor, stimulates endogenous synthesis of nitric oxide or is a substrate for nitric oxide synthase are administered separately.

67. The method of claim 62 or 64, wherein the at least one thromboxane inhibitor and the at least one compound that donates, transfers or releases nitric oxide, elevates endogenous levels of endothelium-derived relaxing factor, stimulates endogenous synthesis of nitric oxide or is a substrate for nitric oxide synthase are in the form of a composition.

68. A method for treating a sexual dysfunction in a patient in need thereof comprising administering to the patient at least one thromboxane inhibitor and at least one vasoactive agent.

69. The method of claim 68, further comprising administering to the patient at least one compound that donates, transfers or releases nitric oxide, elevates endogenous levels of endothelium-derived relaxing factor, stimulates endogenous synthesis of nitric oxide or is a substrate for nitric oxide synthase.

70. A method for treating or method for treating or preventing an ischemic heart disorder, a myocardial infarction, an angina pectoris, a stroke, a migraine, a cerebral hemorrhage, a cardiac fatality, a transient ischaemic attack, a complication following an organ transplant, a coronary artery bypass, an angioplasty, an endarterectomy, atherosclerosis, a pulmonary embolism, bronchial asthma, bronchitis, pneumonia, circulatory shock of various organs, nephritis, a graft rejection, a cancerous metastases, a pregnancy-induced hypertension, a preeclampsia, an eclampsia, a thrombotic or thromboembolic disorder, an intrauterine growth, a gastrointestinal disorder, a renal disease or disorder, a disorder resulting from elevated uric acid levels, a dysmenorrhea, inhibiting platelet aggregation or platelet adhesion or relaxing smooth muscles in a patient in need thereof comprising administering to the patient at least one thromboxane inhibitor and at least one therapeutic agent.

71. The method of claim 70, further comprising administering to the patient at least one compound that donates, transfers or releases nitric oxide, elevates endogenous levels of endothelium-derived relaxing factor, stimulates endogenous synthesis of nitric oxide or is a substrate for nitric oxide synthase.

72. The method of claim 68 or 70, wherein the at least one thromboxane inhibitor and the at least one vasoactive agent or therapeutic agent are administered separately.

73. The method of claim 68 or 70, wherein the at least one thromboxane inhibitor and the at least one vasoactive agent or therapeutic agent are administered in the form of a composition.
